(19) United States
(12) Patent Application Publication
(10) Pub. No.: US 2004/0147466 A1
(43) Pub. Date: Jul. 29, 2004

(54) Nucleic acid delivery formulations

(76) Inventors: Shikha P. Barman, Bedford, MA (US); Daoing Wang, Bedford, MA (US); Mary Lynne Hedley, Lexington, MA (US); Krishnendu Roy, Watertown, MA (US)

Correspondence Address: FISH & RICHARDSON PC, 225 FRANKLIN ST, BOSTON, MA, 02110, US

(21) Appl. No.: 10/466,289

(22) PCT Filed: Jan. 17, 2002
(86) PCT No.: PCT/US02/01379

(51) Int. Cl. A61K 48/00

(52) U.S. Cl. 514/44; 514/12

(57) ABSTRACT

The invention is based on the discovery that injectable and nucleic acid-compatible polymeric compositions and formulations can be structurally designed to regulate nucleic acid activity or gene expression in vivo, for example, by controlling the bioavailability of the nucleic acid via modulation of the biodegradability and crosslink density of the network formed by the components of the formulation. The polymeric network encases the nucleic acid, not only controlling the release of the DNA, but also providing protection from degradation. The invention described herein improves upon prior modes of gene delivery, in that gene expression can be regulated by modulation of a polymeric network formed by combination of at least two water-soluble components capable of reacting with one another. The nucleic acid of interest is incorporated into the network to be released in a sustained manner to achieve level and duration of activity or expression needed.

### BACKGROUND OF THE INVENTION

This invention relates to methods and compositions for delivering nucleic acids to cells. In particular, the invention relates to delivery of nucleic acids for the purpose of gene expression from a bioabsorbable polymeric network structurally and functionally designed to induce gene expression.

Techniques for expression of exogenous DNA molecules hold considerable potential for the treatment of hereditary diseases, e.g. cystic fibrosis. These techniques can also be used when expression of gene products from genes not naturally found in the host cells is desired, for example, from genes encoding cytotoxic proteins targeted for expression in cancer cells. In one application, individuals can be treated with an exogenous DNA that can express a therapeutic polypeptide for some duration (e.g., days, weeks, a month, or several months) as needed for the particular treatment. DNA vaccines can be delivered in these formulations.

The emergence of methods for gene transfer to mammalian cells has prompted enormous interest in the development of gene-based technologies for the treatment of human disease. To date, gene expression technology has focused primarily on the use of viral vectors that provide highly efficient transduction and high levels of gene expression in vivo. The most well-studied vectors are adenoviral vectors, particularly those from replication-defective viruses. These vectors can efficiently transduce non-dividing cells, generally do not integrate into the host cell genome, and can result in high levels of transient gene expression. However, the use of viral vectors has raised safety issues relating to, for example, host response to the virus, and oncogenic and inflammatory effects.

Other, non-viral gene transfer techniques that have been employed include biolistic transfer, injection of “naked” DNA (U.S. Pat. No. 5,580,859), delivery via cationic liposomes (U.S. Pat. No. 5,264,618), and delivery via microparticles (U.S. Pat. No. 5,783,567), delivery via lipofection/liposome fusion products (Proc. Nat'l Acad. Sci., Vol. 84, pp. 7413-7417 (1993), and methods based on the use of polymers that can be admixed with nucleic acids in solution and delivered to muscle tissue (U.S. Pat. No. 6,040,295).

A significant disadvantage of these methods is they usually provide for only transient gene expression, and repeated administrations would thus be necessary if continued gene expression were needed.

### SUMMARY OF THE INVENTION

The invention is based on the discovery that injectable and nucleic acid-compatible polymeric compositions and formulations can be structurally designed to regulate gene expression in vivo, for example, by controlling the bioavailability of the nucleic acid via modulation of the biodegradability and crosslink density of the network formed by the components of the formulation. The polymeric network encases the nucleic acid, not only controlling the release of the DNA, but also providing protection from degradation. The invention described herein improves upon prior modes of gene delivery, in that gene expression can be regulated by modulation of a polymeric network formed by combination of at least two water-soluble components capable of reacting with one another. The nucleic acid of interest is incorporated into the network to be released in a sustained manner to achieve the level and duration of expression needed.

In general, the invention features an injectable aqueous formulation that contains: (a) a nucleic acid; (b) a first non-nucleic acid, water-soluble component; and (c) a second non-nucleic acid, water-soluble component, wherein the first and second components each include two or more reactive groups, the reactive groups of the first component being reactive with the reactive groups of the second component.

The first and second components of the formulation can react with one another to form a branched or a crosslinked polymeric network. The first and/or second components can include one or more succinimidyl, chloroformate, acrylate, amino, alcohol, thiol epoxide, sulfhydryl, or hydrazidyl groups. In one example, at least one of the first and second components is a functionalized multi-armed poly(alkylene oxide) (i.e., a branched poly(alkylene oxide, or a poly(alkylene oxide) having more than one arm (e.g., having eight or 16 arms emanating from a center) such as poly(ethylene oxide), poly(ethylene oxide)-co-poly(propylene oxide)-co-poly(ethylene oxide), poly(propylene oxide)-co-poly(ethylene oxide)-co-poly(propylene oxide). In another example, at least one of the first and second components is a polyethylene glycol tetraamine. In another example, at least one of the first and second components is a polyethylene glycol tetrasuccinimidyl glutarate. In another example, at least one of the first and second components is a polyethylene glycol tetra-sulfhydryl. In another example, at least one of the first and second components is a functionalized poly(alkylene oxide) with at least two reactive functional groups, e.g., an epoxide, aldehyde, pyrophosphate, or any other functional group. In another example, at least one of the first and second components is a polyamidoamine having at least 2, 3, 4, 5, 6, 7, 8, 9, 10, 15, or 20 or more (e.g., 4 to 8 or 10 to 15) reactive functional groups, e.g., amino groups. In another example, at least one of the first and second components is a polyethylimine or polylysine derivative. In another example, at least one of the first and second components is a functionalized chitosan, cyclodextrin, or poly(vinyl alcohol) with at least two reactive functional groups. In another example, one or both of the first and second components includes three or more reactive groups, the reactive groups of the first component being reactive with the reactive groups of the second component.

In one embodiment, a formulation of the invention can further include a third non-nucleic acid, water-soluble component. The third component can optionally include at least one reactive group. In some cases, the reactive group(s) of the third component can be reactive with at least one reactive group of the first component or the second component, with both the first and second components, with a product formed by reaction of the first and second components, or with neither the first nor the second component. In one example, the third component is methoxy-polyethylene glycol-di-stearoyl-phosphatidylethanolamine (PEG-DSPE).

In one embodiment, a formulation of the invention can further include an excipient In one example, the excipient is a neutral, anionic, or cationic lipid. In another example, the excipient is a sugar (e.g., sucrose, dextrose, or trehelose), polyethylene glycol, chitosan, hyaluronic acid, chondroitin sulfate, heparan sulfate, phosphatidyl inositol, glucosamine, polyvinyl alcohol, Pluronics® (BASF, Inc., Mount Olive, N.C. U.S.A.), derivatized Pluronics®, or derivatized polyethylene glycol.

In an example, an excipient includes a permeation enhancer. Examples of “permeation enhancers” include pluronics (e.g., poloxamers), polyethylene glycol, polypropylene glycol, propylene glycol-based molecules, sodium dodecyl sulfate (SDS), poly-vinyl pyrrolidone (PVP), Vitamin E and Vitamin E-tocopherol acetate (e.g., Vitamin E-TPGS®, Eastman Kodak, Inc., Kingsport, Tenn., U.S.A.), lauroyl and oleoyl macrogol glycerides (e.g., Labrafils® and Gattefosse®, both available from Gattefosse, Westwood, N.J., U.S.A.), lipids, glycerol, polyoxyethylene sorbitan monoesters, Tween® 20 and 80, Span® 80, fatty acids, fatty acid esters, bile salts (e.g., taurocholic acid and glycocholic acid), Brij®, sodium hyaluronate (Genzyme Corp, Framingham, Ma., USA), bolaphiles, and sorbitan oleates (Sigma, Inc.).

In another example, the excipient includes a bioavailability enhancer. Examples of “bioavailability enhancers” include propylene glycol and macrogol-based enhancers (e.g., Gelucire® (Gattefosse), Labrafil® (Gattefosse), Capryol® (Gattefosse), Labrasol® (Gattefosse), Plurol® (Gattefosse)), Bioperine® (Sabinsa Corporation, New Jersey, U.S.A.), Vitamin E (Sigma, Inc.)and Vitamin E-TPGS® (Eastman Kodak), poloxamers such as Pluronics® (BASF, Inc.), and polyethylene glycol (Sigma, Inc.).

In another example, the excipient is a protein (e.g., contains a cytokine).

In another example, the excipient contains a small molecule drug, e.g., an anti-tumor agent, anti-neoplastic, anti-inflammatory, or antibiotic.

In another example, the excipient is an adjuvant (e.g., a CpG oligonucleotide, oil, lipid, monophosphorolipid (MPL; Sigma, Inc.), lipopolysaccharide(LPS; Sigma, Inc.), or carbohydrate).

In another example, the excipient is chemically bound to the crosslinked polymeric network or branched polymer, e.g., methoxy-PEG-monoamine, distearoylethanolamine, stearylamine, spermine, spermidine, laurylamine, urea, dioleylethanolamine, or aminocaproic acid. All of these excipients are reactive with the network, forming covalent bonds. In another example the excipient contains a component that stabilizes a nucleic acid, e.g., sodium, calcium, zinc, or magnesium salts of bicarbonates.

An example of a reactive excipient is phosphatidyl ethanolamine, which can react with poly(ethylene oxide)-tetrasuccinimidyl glutarate (P4-SG). Another example of a reactive excipient is a poly(amino acid) containing multiple cysteines in its backbone (e.g., poly(cysteine) or a peptide such as poly(arg-lys-cys-guanine-arg-cys-cys-lys-cys)). The free —SH of the cysteines can would react easily with P4-SG. Another example is poly(lysine), with the pendant amino groups of which can reacting easily with P4-SG. When P4-SG and/or poly(ethylene glycol)-tetraamine (P4-AM) are components of the new formulations, Just cysteine or lysine can also be used as an excipient.

In another aspect, the invention features an injectable aqueous formulation that contains: (a) a nucleic acid; (b) a first non-nucleic acid, water-soluble component; (c) a second non-nucleic acid, water-soluble component, and (d) a third non-nucleic acid, water soluble component, wherein the first, second and third components each include two or more reactive groups, the reactive groups of the third component being reactive with the reactive groups of the first component or the second component.

A formulation can include more than one species of nucleic acid, e.g., two or more species of nucleic acids, each encoding a different polypeptide or a nucleic acid encoding a polypeptide and an oligonucleotide. In addition, a nucleic acid can be an oligonucleotide (e.g. with a phosphodiester or phosphorothioate backbone). In one example, a nucleic acid encodes a therapeutic protein or a protein that induces an immune response. As used herein, a “therapeutic protein” is a protein that when administered to an individual confers a therapeutic benefit upon the individual. By “protein that induces an immune response” is meant a pathogenic protein (e.g., a viral or bacterial protein) or portion thereof, a tumor-associated antigen or portion thereof, or another protein that is involved in disease (e.g., a neurodegenerative (e.g., Alzheimer's), cardiac, immunologic, autoimmune, or gerontologic disease).

A nucleic acid of a formulation described herein can be in any form, e.g., a solution, dispersion, powder, precipitated, condensed, micronized, or emulsion. A nucleic acid can optionally be encapsulated in or associated with a biodegradable polymeric microparticle. Examples of useful microparticles are described in U.S. Pat. No. 5,783,567, U.S. Ser. No. 09/909,460 (which is a continuation of U.S. Ser. No. 09/321,346), and U.S. Ser. No. 09/872,836, the contents of which are incorporated by reference in their entirety. In one example, the nucleic acid is released from the branched or crosslinked polymeric network by biodegradation or by simple diffusion.

In one embodiment, a formulation described herein forms a hydrogel at a temperature between about 20° C. and about 40° C. within about 20 minutes after the formulation is prepared. In other embodiments, a formulation described herein forms a hydrogel at a temperature between about 25, 30, 35, or 37° C. and about 40° C., within about 19, 18, 17, 16, 15, 14, 13, 12, 11, 10, 9, 8, 7, 6, 5, 4, 3, 2, 1, or less than 1 minute after the formulation is prepared. In one example, a formulation described herein remains injectable for at least fifteen seconds, e.g., at least 15, 30, 45, 60, 90, 120, 180, 240, 300, or 600 seconds, or 15 minutes or 20 minutes after the formulation is prepared.

In another embodiment, the network of a formulation described herein forms a viscous liquid.

In another embodiment, the nucleic acid is protected from serum nucleases by incorporation into the network. In one example, the nucleic acid is expressed following injection of the network (e.g., into muscle). In another example, an immune response is generated to the nucleic acid encoded antigen following injection of the network/nucleic acid formulation. In another example, the release of the nucleic acid following injection is controlled by the cross-linking density of the network. In another example, the expression of the nucleic acid following injection is controlled by the cross-linking density of the network.

The first and/or second components of a formulation can be biodegradable, e.g., by a hydrolytic or proteolytic mechanism. The network of formulation can be biodegradable, e.g., by a hydrolytic or proteolytic mechanism.

The branched or crosslinked polymeric network, e.g., fully or partially crosslinked, of a composition can include linkages selected from the group consisting of ester, carbonate, imino, hydrazone, acetal, orthoester, peptide, amide, urethane, urea, amino, oligonucleotide, and sulfonamidyl bonds. As used herein, “partially” means that the stoichiometry of the components can be adjusted, so that some of the functional groups remain unreacted, e.g., to obtain a loose network. As used herein, “fully” means that the stoichiometry of the components is equimolar, e.g., essentially all available functional groups have been reacted in the network.

The first and/or second components of a formulation can include one or more sulfhydryl, amine, epoxide, phosphoroamidates, chloroformate, acrylate, carboxylic acid, aldehyde, succinimide ester, succinimide carbonate, maleimide, iodoacetyl, carbohydrate, isocyanate, and/or isothiocyanate groups.

At least one of the first and second components of a formulation described herein can include a biodegradable linkage such as a lactate, caproate, methylene carbonate, glycolate, ester-amide, ester-carbonate, or a combination thereof.

In another embodiment, the formulations described herein can be in the form of microparticles (e.g., microparticles, nanoparticles, microspheres, or nanospheres). Such microparticles can be essentially “solid,” meaning that the cross-linked polymer (e.g., the hydrogel) formed by reaction of the first and second non-nucleic acid components can be distributed, evenly or unevenly, throughout each microparticle, with the nucleic acid distributed within the three-dimensional structure of the polymer. Alternatively, the microparticles can have outer shells made up of the cross-linked polymer, and the nucleic acid can be either within the polymeric structure or else in the core of the microparticle. The invention also features a method for making such microparticles. The method includes introducing the nucleic acid and the first and second non-nucleic acid components of any of the formulations described herein into an emulsifying bath (e.g., a homogenizer or blender, or other device capable of emulsifying a mixture), either separately or after combining, and then emulsifying (e.g., by homogenizing or blending) the resulting mixture in the emulsifying bath for at least part of the time that the first and second non-nucleic acid, water-soluble components are reacting with each other. By adjusting the concentrations, ratios, emulsification speed, and identities of the components, the size, structure, and other physical properties of the microparticles can be controlled. For example, microparticles smaller than about 500 microns, 250 microns, 100 microns, 50 microns, 20 microns, 15 microns, 10 microns, 5 microns, 2 microns, 1 micron, or still smaller can be prepared. Generally, higher homogenization rates result in smaller microparticles.

In another aspect, the invention includes a method of making a polypeptide by applying a formulation described herein to a cell. The nucleic acid contained within the formulation can code for expression of the polypeptide. In one example, the formulation is applied to a cell within an animal, e.g., administered to the animal by injection, extrusion, or spraying.

In another embodiment, the invention includes a method of making a polypeptide by injecting into an animal, e.g., a mouse, rat, pig, non-human primate, or human, a formulation described herein. The nucleic acid contained within the formulation can code for expression of the polypeptide. In one example, the formulation is injected in, on, or adjacent to a tumor. In another example, the formulation is injected intramuscularly, subcutaneously, or intra-joint. The formulation can be injected into the animal once or more than once. The formulation can be delivered, for example, via an aerosolizer or nebulizer. The formulation can alternatively be applied to the skin, delivered in a patch, or placed on a wound. The formulation is also suitable for delivery via needle-free devices. The formulation can be delivered by any of these mechanisms and then followed by an electrical pulse. Electrical pulses are known to enhance uptake of macromolecules post injection as described in U.S. Pat. No. 5,993,434. The formulation can be premixed before injection.

In another aspect, the invention includes a method of producing a polypeptide by: (a) providing a surface suitable for cell culture; (b) adding a formulation of the invention to the surface; and (c) placing a cell on the formulation. According to this method, the nucleic acid codes for expression of a polypeptide, and the cell produces the polypeptide following the culturing of the cell in vitro.

In another aspect, the invention features a method of making a dried nucleic acid formulation by: (a) preparing a mixture by mixing in an aqueous solution (i) a nucleic acid, (ii) a first non-nucleic acid, water-soluble component, (iii) a second non-nucleic acid, water-soluble component, and (iv) a third non-nucleic acid, water-soluble component, wherein the first and second components each include two or more reactive groups, the reactive groups of the first component being reactive with the reactive groups of the second component at a pH greater than 7.0. Optionally, the third component can include at least one reactive group that is reactive at a pH greater than 7.0 with at least one reactive group of the first component, the second component, of both the first and the second components, of a product formed by reaction of the first and second components, or with neither the first nor the second component. The aqueous solution has a pH and/or temperature that prevents the first and second components from reacting to form a crosslinked network (i.e., at a pH lower than 7.0); and (b) drying the mixture to thereby create a dried formulation (e.g., drying in a lab dryer under vacuum, or in a lyohilizer). This method permits the preparation of a formulation that contains unreacted components in a single vessel, e.g., the method avoids the necessity of maintaining the components in separate vessels until just prior to initiating a crosslinking reaction. The mixing step can be performed at a pH less than about 7.0, e.g., less than about 6.0. In one example, the mixing step is performed at a pH of about 5.5. The mixing step can be performed at or below about 4° C., e.g. between 0° C. and 4° C. In one example, the mixture is dried. In another example the mixture is lyophilized.

In another embodiment, both components can be individually dried (optionally with nucleic acid and/or excipient in one or both of the components), and then a buffer is added to reconstitute the formulation.

In one embodiment, the first non-nucleic acid, water-soluble component is polyethylene glycol amine. In another embodiment, the second non-nucleic acid, water-soluble component is polyethylene glycol succinimidyl glutarate. In another embodiment, the first non-nucleic acid, water soluble component is polyethylene glycol sulfhydryl. In another embodiment, the third non-nucleic acid, water-soluble component is methoxy-polyethylene glycol-di-stearoyl-phosphatidylethanolamine (PEG-DSPE).

The invention also features a method of preparing a gel-forming nucleic acid formulation. The method entails adding a buffer having a pH greater than 7.0 to a dried nucleic acid formulation of the invention. The addition of the buffer results in the formation of a crosslinked network between the first and second components. For example, the buffer can be a phosphate buffer with a pH of about 7.5. The buffer can include nucleic acid and/or excipients (e.g., sucrose, Tris, EDTA). The adding step can be performed at or above 20° C., e.g., at or above 37° C.

In another aspect, the invention features a dried nucleic acid formulation that contains: (a) a nucleic acid; (b) a first non-nucleic acid, water-soluble component; (c) a second non-nucleic acid, water-soluble component; and (d) a third non-nucleic acid, water-soluble component. The first two components, and optionally the third component as well, each include two or more reactive groups, the reactive groups of the first component being reactive with the reactive groups of the second component or with the reactive groups of the third component, or both, and/or the third component also includes at least one reactive group that is reactive with at least one reactive group of the first component, with at least one reactive group of the second component, with at least one reactive group of each of the first and second components, with a reactive group of the product formed by reacting the first and second components, or with neither the first nor the second component. In the dried formulation, the three components are each in an unreacted state, and the nucleic acid and the three components are not in solution. The formulation can be lyophilized. In one embodiment, the first non-nucleic acid, water-soluble component is polyethylene glycol amine. In one embodiment, the first non-nucleic acid, water-soluble component is polyethylene glycol sulfhydryl. In still another example, the second non-nucleic acid, water-soluble component is polyethylene glycol succinimidyl glutarate. In another embodiment, the third non-nucleic acid, water-soluble component is methoxy-polyethylene glycol-di-stearoyl-phosphatidylethanolamine (PEG-DSPE).

The invention also features a kit containing a dried formulation described herein; and a buffer having a pH of at least 7.0.

The invention also includes a method of administering a nucleic acid to an individual by: preparing a mixture by adding a buffer having a pH of at least 7.0 to a dried formulation described herein; incubating the mixture to permit the formation of a crosslinked network; and administering the mixture to the individual.

As used herein, a “nucleic acid” can be either RNA or DNA, including, for example, cDNA, genomic DNA, oligonucleotides, mRNA, viral DNA, bacterial DNA, plasmid DNA, triplex nucleic acid, peptide-nucleic acid (PNA) formulations, or condensed DNA. In a preferred embodiment, the nucleic acid is plasmid DNA. In another embodiment, the nucleic acid is an oligonucleotide. The oligonucleotide can include stabilizing features such as base or backbone modifications (e.g., phosphorothioate backbone). The oligonucleotide can be an antisense oligonucleotide, utilized to treat various diseases. In one example, the oligonucleotide can have anti-tumor activity. In yet another example, the oligonucleotide can be used as an adjuvant, e.g., as described in EP 01005368 and WO 99/61056.

By “bioavailability of the nucleic acid” is meant that the delivery formulation prolongs availability of the nucleic acid. By altering the polymer formulation, one can increase or decrease the rate of release of nucleic acid from the polymer network, in turn affecting activity or expression levels. In some embodiments, the polymeric network is biodegradable, i.e., it breaks down into components that are readily cleared from the body.

By “modulation of the polymeric network” it is meant, for example, that the hydrolytically labile linkages can be varied in length and type to affect the degradation time of the network. A fast-degrading polymeric network would provide a higher bioavailability of the nucleic acid to the target cells than would a slower degrading network. Alternatively, excipients can be added to enhance breakdown of the network For example, succinimidyl propionates, succinimidyl caproate or succinimidyl carbonates can be substituted for succinimidyl glutarate in a PEG component to lower the rate of hydrolytic degradation of the network. Conversely, sulfhydryls can be substituted for amines in a PEG component to increase the rate of hydrolytic degradation of the network.

In another example of modulation of the polymeric network, the concentration of the gel-forming components can be varied to change the nature of the network. Thus, for example, a higher concentration of these components results in longer degradation times and increased branching and/or cross-linking, leading to a lower availability of the nucleic acid to the target cells and, consequently, a lower expression level or a lower level of therapeutic nucleic acid.

In still another example of modulation of the polymeric network, the molecular weight of the gel-forming components can be selected to vary the nature of network, particularly the molecular weight between cross-links. A tighter network can result from the use of lower molecular weight components, causing greater retention of the DNA at the site and, consequently, sustained release for a longer duration. By “sustained release”, it is meant that the nucleic acid is available to the target cells for uptake for a longer period of time than would be achieved if the administration of the nucleic acid were in, for example, saline, from which fast dissipation of the DNA from the site would occur.

In yet another example of modulation of the polymer network, addition of a third polymer to one of the pre-formulation components, to form either an interpenetrating network or a semi-interpenetrating network, can vary the nature of the network to control release of DNA at the site to control level and duration of expression. Examples of suitable “third polymers” include methoxypolyethylene oxide-monoamine, polyethylene glycol, poloxamers, and methoxypolyethylene oxide-distearoyl ethanolamine and 8-, 16-, and 32-arm derivatized and non-derivatized polyethylene oxide. In another embodiment, the invention features a method of delivering a particle to an individual. The method includes administering to the individual a formulation that includes: (1) the particle; (2) a first non-nucleic acid, water soluble component; and (3) a second non-nucleic acid, water soluble component. The first and second components each include two or more reactive groups, the reactive groups of the first component being reactive with the reactive groups of the second component. The particle can be, for example, a virus or viral particle or a virus-like particle (VLP) (e.g., adenovirus or adenoviral particle such as an aviadenovirus or mastadenovirus or a penton, hexon, capsid, or other fragment thereof, or VLP made of hepatitis, or papillomavirus components).

By “excipient” is meant a molecule added for the purpose of enhancing or sustaining DNA uptake, activity, or expression, or to further enhance DNA stability, or to modulate release of DNA or degradation of the network In certain embodiments, the excipient is a bioavailability enhancer. By “bioavailability enhancer” is meant an excipient that improves or enhances bioavailability of the DNA to the target cells by its retention at the cell site.

The invention provides several advantages. For example, the new methods and formulations feature an injectable polymer-based slow release system that can afford sustained systemic protein expression (e.g., by delivering genes into skeletal muscles). Such a system can be used in the treatment of “chronic” diseases where multiple administrations are necessary to maintain therapeutic levels of bioactive proteins and peptides. Sustained gene delivery can in turn allow for long-term protein expression. In vitro release experiments described herein indicate that plasmid DNA is slowly released over time from the crosslinked network formulations, with higher crosslinked hydrogels releasing the DNA more slowly. The formulated plasmid DNA generated longer-term protein expression compared to unformulated “naked” DNA in both immunocompetent and complement deficient animals.

The new formulations can also provide protection to the entrapped DNA. Combined with plasmid stability, the new formulations can significantly increase the duration of protein expression following a single administration of DNA; genetic approaches can generate longer protein expression since the intracellular half-life of plasmid DNA is generally much longer than the serum half-life of recombinant proteins. The expression kinetics can be further prolonged by slowly releasing the plasmid over time so that the source of the protein is bio-available for several weeks.

Another advantage of the new formulations of the invention is that they are injectable. Polyethylene glycols are considered to be biomimetic and hence highly biocompatible. They have also been shown to generate minimal inflammation and immune response. Moreover, the new formulations are injectable following reconstitution and do not require surgical implantation procedures.

Furthermore, the crosslinked networks of the invention are readily biodegradable due to the presence of, for example, hydrolytic ester linkages on the P4-SG component. The network components can have a molecular weight on the order of about ˜10,000 Daltons; upon degradation the components can be cleared from the body quite readily.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a chemical structure of certain network components and schematic representation of the crosslinking reaction. The amine and succinimidyl groups react to generate amide linkages between the polymer species thereby forming the network structure. The hydrolytically labile ester linkages in the P4-SG render the network biodegradable.

FIGS. 2A to 2C are graphs depicting network characterization by gel permeation chromatography (GPC) and viscometry. FIG. 2A is a graph of GPC analysis of formulation A. Individual PEG components (P4-SG and P4-AM) are indicated by arrows as is the resulting network, FIG. 2B is a graph of formulations A, B, C and D that were analyzed by viscometry. Data were collected and plotted at different intervals after mixing the two PEG components using the Brookefield Wingather™ software. Viscosity was measured at 37° C. (formulation A) and 25° C. (formulations B, C and D), FIG. 2C is a graph of gelation time (y-axis) plotted as a function of gel concentration (x-axis).

FIG. 3 is a table summarizing the physico-chemical characteristics of network formulations A (2% w/v P4-AM/P4-SG), B (3% w/v P4-AM/P4-SG), C (4% w/v P4-AM/P4-SG) and D (5% w/v P4-AM/P4-SG), detailing appearance of gels, gel swelling and gelation times determined at 25° C. and 37° C.

FIG. 4 is a picture of a gel showing chemical compatibility of pDNA with network components. In lane 1 is a secreted embryonic alkaline phosphatase (SEAP) plasmid, in lane 2 is 1 μg/ml of DNA incubated with 2% w/v (P4-AM+P4-SG); and in lane 3 is 1 μg/ml of DNA incubated with 5% w/v (P4-AM+P4-SG).

FIGS. 5A and 5B are two plots of the swelling properties of 5% (grey), 8% (black), and 10% (white) PEG hydrogels formulated at different intervals following stock solution preparation to examine solution stability. Overnight swelling (percent increase in weight) was performed at 37° C. in phosphate-buffered saline (PBS) with blank PEG-hydrogels (A) or with hydrogels containing plasmid and mPEG-DSPE (B).

FIG. 6 is a table depicting the injectability of P4-AM/P4-SG formulations. Maximum time for injection (min) after reconstitution is shown for formulations A (2% w/v P4-AM/P4-SG), B (3% w/v P4-AM/P4-SG), C (4% w/v P4-AM/P4-SG) and D (5% w/v P4-AM/P4-SG).

FIG. 7A is a graph showing in vitro release of plasmid from network formulations as measured by HPLC analysis. Depicted is a typical HPLC trace of DNA released from formulation C. The first peak represents polyethylene glycol, the second set of peaks (triplet) represents different isoforms of plasmid (supercoiled plasmid is represented by the second peak).

FIG. 7B is a graph depicting cumulative release of DNA from formulations B, C, and D at different time intervals (days post administration). Day 1 is represented by the white bar, day 3 by the black bar, day 7 by the stippled bar, and day 14 by the grey bar.

FIG. 8 is a picture of a gel depicting protection of network entrapped DNA from serum digestion. In lane 1 is unformulated DNA, no serum; in lane 2 is formulation A, no serum; in lane 3 is formulation B, no serum; in lane 4 is unformulated DNA+1:40 serum; in lane 5 is formulation A+1:40 serum; in lane 6 is formulation B+1:40 serum; in lane 7 is unformulated DNA+1:80 serum; in lane 8 is formulation A+1:80 serum; and in lane 9 is formulation B+1:80 serum. The arrow represents supercoiled DNA.

FIG. 9 is a graph depicting the swelling properties of 10% w/v P4-SG/0.5% w/v poly(amidoamine) (PAMAM) hydrogels. Swelling was tested following overnight incubation of gels at 37° C. (n=3). Gels were formed in the presence (w/mPEG-DSPE) or absence (w/o mPEG-DSPE) excipient.

FIG. 10 is graph depicting the analysis of gel times for P4-SG/poly(ethylene oxide)-sulfydryl (P4-SH) networks. Viscosity was measured at 250° C. for 3%, 4%, and 10% w/v PEGs formulations. Symbols for each gel formulation are indicated. Y-axis represents viscosity (cp) and the x-axis represents time (minutes). Data were collected at different intervals after mixing the two PEG components using the Brookefield Wingather™ software.

FIGS. 11A and 11B are graphs depicting the expression of SEAP in mice injected with network containing SEAP DNA. FIG. 11A shows serum SEAP level indicated on the y-axis (ng/ml) and the formulation is indicated on the x-axis. Time points are indicated by different filled bars. FIG. 11B shows the percent of animals within each group that express more than 300 pg/ml serum SEAP (y-axis) at days 10 (black bars), 33 (striped bars), and 92 (white bars), as indicated for each formulation (x-axis).

FIGS. 12A and 12B are graphs depicting the SEAP expression in complement deficient DBA/2 mice. FIG. 12A show the percent of SEAP expressing animals (animals expressing >300 pg/ml at a given time point) as indicated on the (y-axis). Mice were injected with the SEAP DNA containing formulation groups indicated on the (x-axis). Timepoints are as indicated (days 7, 35, 81). FIG. 12B shows expression of serum SEAP in RAG2 immunocompromised mice injected with P4-AM/P4-SG networks containing SEAP plasmid DNA. Percent of SEAP expressing animals (animals expressing >300 pg/ml at a given time point) is indicated on the (y-axis). Mice were injected with the SEAP DNA containing formulation groups indicated on the (x-axis). Timepoints are as indicated (days 7, 14, 30 and 42).

FIG. 13 is a graph depicting the effect of electroporation on serum SEAP levels. Mice were injected with a GT20 P4-AM/P4-SG formulation. Half the animals received electroporation treatment (“+EP”) as depicted on x-axis. Serum SEAP level is indicated on the y-axis (ng/ml). Serum samples were tested 7 days post administration in mouse muscle.

FIGS. 14A and 14B are graphs depicting how serum SEAP expression can be influenced by network containing excipients. FIG. 14A shows serum SEAP levels (ngs/ml) as indicated on the y-axis. Excipients formulated with the GT20 network are indicated on the x-axis. The bars represent the following excipients formulated with GT20 networks, respectively: GT20+0.1% SDS, GT20+0.1% L62, GT20+0.15% PAMAM. FIG. 14B is a graph showing GT20+0.025% w/v Streptolysin, GT20+250 mg/ml, Magainin I, GT20 with no excipient. Serum samples were tested 7 days post administration in mouse muscle.

FIG. 15 is a graph depicting how SEAP expression is mediated by DNA in P4-SH/P4-SG networks. Serum SEAP levels (ngs/ml) are indicated on the y axis and the formulation is indicated on the x-axis (3.5% w/v, formulation A; 5% w/v, formulation B). Serum samples were tested 7 days post administration.

FIG. 16 is a graph depicting how β-gal specific antibody is elicited in mice immunized with network formulated DNA. Titers of β-gal specific IgG from pooled serum samples (n=4) were determined 12 weeks post injection. Titers from individual serum are indicated on the y-axis and the formulation is indicated on the x-axis. The data are presented as the mean±standard error (SE) of four mice performed in duplicate. *, p=0.014 formulation A vs saline control; **, p<0.01 formulation B vs saline and p=0.137 formulation A vs formulation B by Student's t test.

FIG. 17 is a graph depicting T cell proliferative β-gal specific responses in mice immunized with formulated DNA. Responses are from pooled samples (n=4) at 12 weeks post-immunization. Antigen used in the stimulation is indicated (β-gal (dark bars) or chicken ovalbumin (OVA) (white bars) protein). The immunizing formulation is indicated on the x-axis and the stimulation index (SI, the median counts per minute (cpm) of the maximum response to antigen divided by the median cpm in the absence of antigen) is indicated on the y-axis. Data are expressed on the y-axis as mean of triplicate samples±SE. *, p=0.01 formulation A vs saline; **, p<0.001 formulation B vs saline and p=0.26 in comparison between two formulations.

FIG. 18 is a graph depicting interferon gamma Elispot analysis of T cells in mice immunized with network formulated DNA. Splenic T cells were harvested at 12 weeks post-immunization and pooled (n=4). Response to H-2L^{d} restricted, β-gal 876-884 peptide (filled bars) or HBV peptide (hatched bar) or media (open bar) is indicated. The number of IFN-γ+spot forming cells/10⁶ T cells is indicated on the y-axis. The relevant formulation (A or B) and untreated control are indicated on the x-axis. The data are presented as the mean±SE of four mice performed in triplicate. *, p<0.001 in comparison with saline control; p=0.52 formulation A vs formulation B.

FIG. 19 is a table depicting protection of mice immunized with network formulated DNA. BALB/c mice were challenged by i.v injection of either 5×10⁶ CT26.WT or β-gal expressing CT26.CL25 tumor cells, three weeks post immunization. The number of tumor nodules is indicated in each group.

FIG. 20. is a schematic depicting a method of preparing a lyophilized formulation that can be reconstituted in a single vial prior to use.

FIG. 21 is a graph depicting how lyophilization does not effect gel time. Viscosity of a 10% w/v P4-SH/P4-SG network formulation that was not-lyophilized compared to a reconstituted lyophilized formulation. Symbols for each formulation are indicated. The y-axis represents viscosity (cp) and x-axis represents time (minutes). Data were collected at different intervals after mixing the two PEG components using the Brookefield Wingather™ software.

FIG. 22 is a graph depicting interferon gamma Elispot analysis of T cells in mice immunized with lyophilized or non-lyophilized formulations. Formulations included 2% w/v P4-AM/P4-SG and 3% w/v P4-AM/P4-SG. Splenic T cells were harvested at 12 weeks post-immunization and individually analyzed (n=4). Mean responses to H-2L^{d} restricted, β-gal 876-884 peptide (filled bars) or HBV peptide (hatched bar) or media (open bar) are indicated. The number of IFN-γ spot forming cells/10⁶ T cells is indicated on the y-axis. The relevant formulations administered as reconstituted lyophilized or non-lyophilized formulations, and a saline control are indicated on the x-axis. p<0.001 in comparison with saline control; p=0.156 for 3% lyophilized vs unlyophilized formulations and p=0. 137 for 2% lyophilized and non-lyophilized formulations.

FIGS. 23A and 23B are graphs depicting release of oligonucleotide from P4-SG/P4-AM gels. Release assays were performed for oligonucleotide in 5% w/v (a) and 10% w/v formulations (b) (x-axis). Release was carried out in phosphate buffered saline, pH 7.4 at 37° C., with n=3 per timepoint. Percent of oligo released within each time frame is indicated on the y-axis.

FIGS. 24A and 24B are graphs depicting how viscosity of a 10% w/v P4-SH/P4-SG network formulation varies with temperature and pH. The y-axis represents viscosity (cp) and the x-axis represents time (minutes). In FIG. 24A, viscosity was performed at 25° C. or 37° C. as indicated. In FIG. 24B, viscosity measurements were performed at various pHs as indicated. Data were collected at different intervals after mixing the two PEG components using the Brookefield Wingather™ software.

FIGS. 25A and 25B are graphs depicting oligonucleotide release from P4-SH/P4-SG gels. The release study was carried out in phosphate buffered saline, pH 7.4 at 37° C., with n=3 gels per timepoint. In FIG. 25A, the y-axis indicates percent oligo released and the x-axis represents the time frame. In FIG. 25B, cumulative oligonucleotide release (y-axis) is plotted versus time (x-axis). Release was performed on 10, 20 and 30% gels as indicated.

FIG. 26 is a graph depicting oligonucleotide release from 10% w/v P4-SG/PAMAM, G0 gels. The release study was carried out in phosphate buffered saline, pH 7.4 at 37° C., with n=3 gels per timepoint. The y-axis indicates percent oligo released and the x-axis represents the time frame.

### DETAILED DESCRIPTION OF THE INVENTION

This invention relates to methods and compositions for delivering nucleic acids to cells. These methods and compositions can be used for a variety of functions including but not limited to the induction of cell activation, the regulation of gene expression, or the induction of gene expression. A nucleic acid is released from a bioabsorbable polymeric network structurally and functionally designed to enhance and optimize the level and duration of the released nucleic acid activity or expression.

The composition of the delivery system includes a polymeric network formed by the chemical combination of at least two injectable non-nucleic acid polymeric components, containing one or more nucleic acids and one or more excipients.

The components (1, 2, and optionally 3) are water-soluble and are composed of polymeric backbones modified to have end functional groups capable of reacting with one another. The reactive functional groups of component 1 can be, for example, chloroformates, acrylates, amines, alcohols, tetrasulfydryls, epoxides, sulfhydryls, hydrazides, or combinations thereof, in the same molecule. The reactive functional groups of component 2 can be, for example, chloroformates, acrylates, carboxylic acids, aldehydes, maleimides, iodoacetyl, carbohydrates, isocyanates, or isothiocyanates. The polymeric network can include linkages such as esters, carbonates, imines, hydrazones, acetals, orthoesters, peptides, amides, urethanes, ureas, amines, oligonucleotides, or sulfonamides. The components can be modified to include biodegradable linkages such as lactates, caproates, methylene carbonates, glycolates, ester-amides, ester-carbonates, or combinations thereof.

The following are examples of the practice of the invention. The examples demonstrate examples of various polymer networks for formulation, characterization and modulation to optimize gene expression levels and duration. They are not to be construed as limiting the scope of the invention in any way.

### EXAMPLES

### Example 1

### In-Situ Formation of Polyethylene Oxide-Polyethylene Oxide Networks via Formation of Amide Linkages

Reacting Polymers

Component 1: Polyethylene oxide-tetraamine (P4-AM), (SunBio Systems, Korea)

Component 2: Poly(ethylene oxide 3350)-tetrasuccinimidyl glutarate (P4-SG), (SunBio Systems)

Polymer Characterization

The degree of substitution (d.s.) of amines on the tetra-armed polyethylene oxide backbone was calculated to be 3.91 by ¹H-NMR; d.s. of succinimidyl glutarate was 3.85, also by ¹H-NMR.

Preparation of Formulations

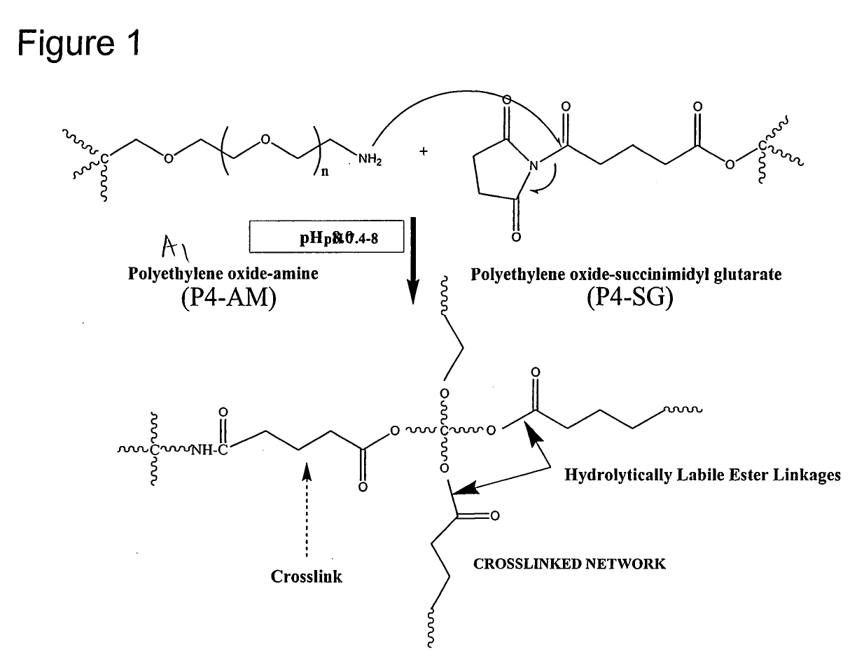

All formulations were prepared by mixing of two solutions, one containing a pre-weighed amount of P4-AM dissolved in 0.1M potassium phosphate buffer, pH 8.0 and the other containing an equimolar amount of P4-SG dissolved in cold deionized water containing nucleic acid (e.g. plasmid or oligonucleotide). All formulations contained 1 mg/ml of the nucleic acid. FIG. 1 shows the chemical structure of the network components (P4-AM/P4-SG) and a schematic representation of the cross-linking reaction by formation of amide linkages. The network is rendered biodegradable by the presence of ester linkages in one of the components, P4-SG.

Formulation of 2%-15% Polymeric Networks

Solutions of the gel-forming components were prepared (2%, 3%, 4%, 5%, 8%, 10% and 15% w/v). For example, 5% P4-AM was prepared by dissolving 50 mg P4-AM in 1 ml potassium mono-di phosphate buffer (pH 8.0). 5% w/v P4-SG was prepared by dissolving 50 mg of P4-SG in milliQ de-ionized water. This solution was stored on ice until use. The networks were created following addition of a solution of P4-AM with P4-SG. For example, Formulation A contained 2% w/v solids. Formulation A cross-linked into a viscous branched polymer. Formulations B-G included equimolar amounts of the same components as in Formulation A, but at higher concentrations (B, 3% w/v total polymer; C, 4%; D, 5%; E, 8%, F, 10% and G, 15%). Formulations B-G cross-linked into tissue-conforming hydrogels in situ, post-injection into muscle.

Incorporation of Plasmid DNA into the Network, and Effect on Gel Time

DNA was added to the solution containing P4-SG. 11.1 μl of a 9 mg/ml stock solution of the nucleic acid (e.g., plasmid DNA or oligonucleotide) was added to 38.8 μl of 6.4% solution of P4-SG, to obtain a 5% P4-SG solution containing 100 μg nucleic acid in 100 μl. 50 μl of this P4-SG/DNA solution was then added to 50 μl of 5% w/v P4-AM to formulate the desired gel of final concentration (5% total PEGs). The gel time of this formulation was approximately the same as a formulation that did not contain nucleic acid (5-6 minutes at 25° C.).

Incorporation of a Third non-Nucleic Acid Polymeric Reagent, and the Effect on Gel Time

A third non-reacting, non-nucleic acid polymeric component was added to the formulation. Methoxy-PEG2K-di-stearoyl-phosphatidylethanolamine (mPEG-DSPE, Genzyme) was selected as a polymeric excipient and added to the solution of P4-SG. To obtain a 10:1 mass ratio of DNA to PEG-DSPE, in a formulation containing 100 μg nucleic acid in 100 μl of gel, 1 μl of a 10 mg/ml mPEG-DSPE solution (in milliQ de-ionized water) was added to 38.7 μl of 6.5% P4-SG and 11.1 μl of a 9 mg/ml nucleic acid solution prior to the addition of P4-AM. The final concentration of the gel was 5% w/v reacting polymers (P4-AM+P4-SG). Gel formation was noted after mixing of the solution. Addition of mPEG-DSPE to the formulation did not alter the gel time at 25° C.

Network Characterization by Gel Permeation Chromatography

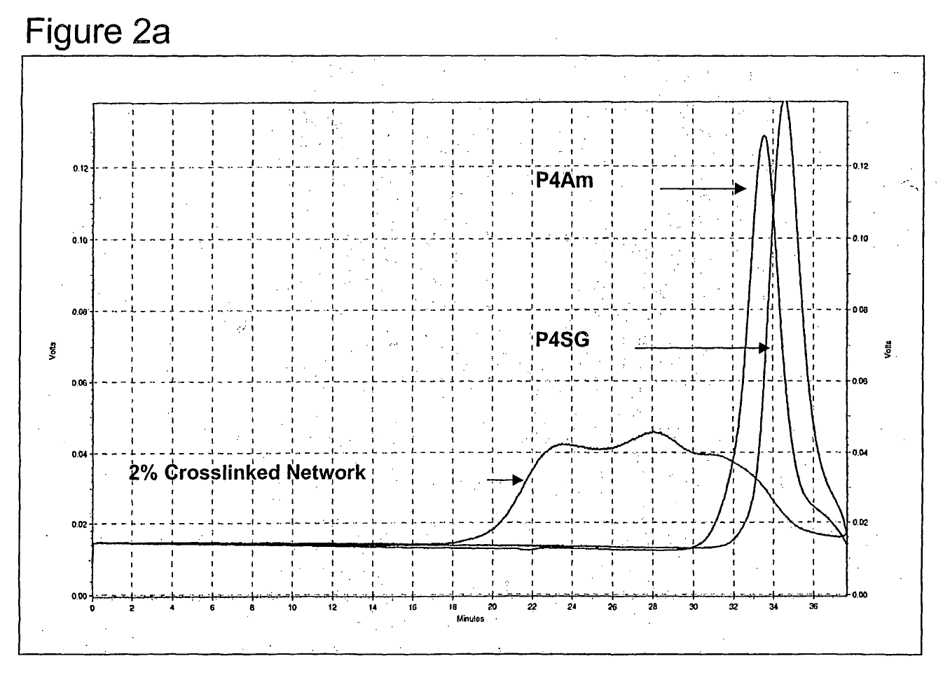

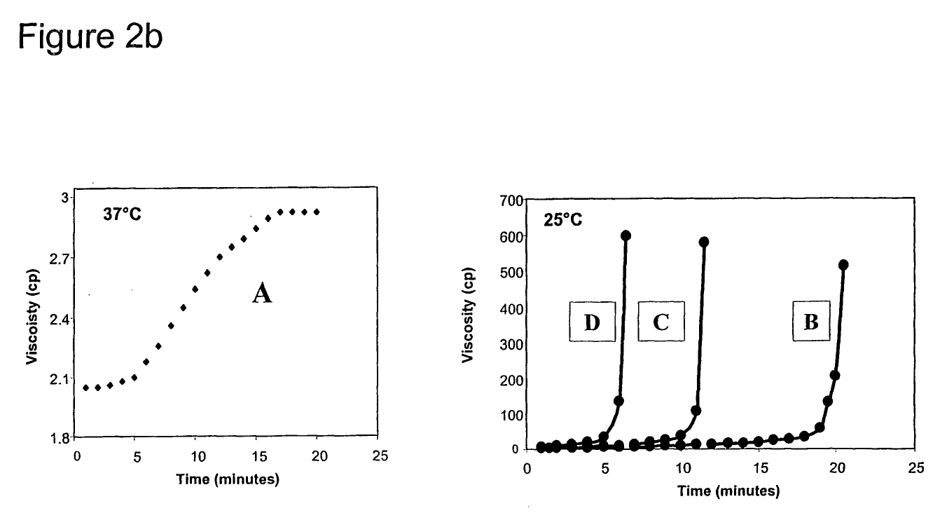

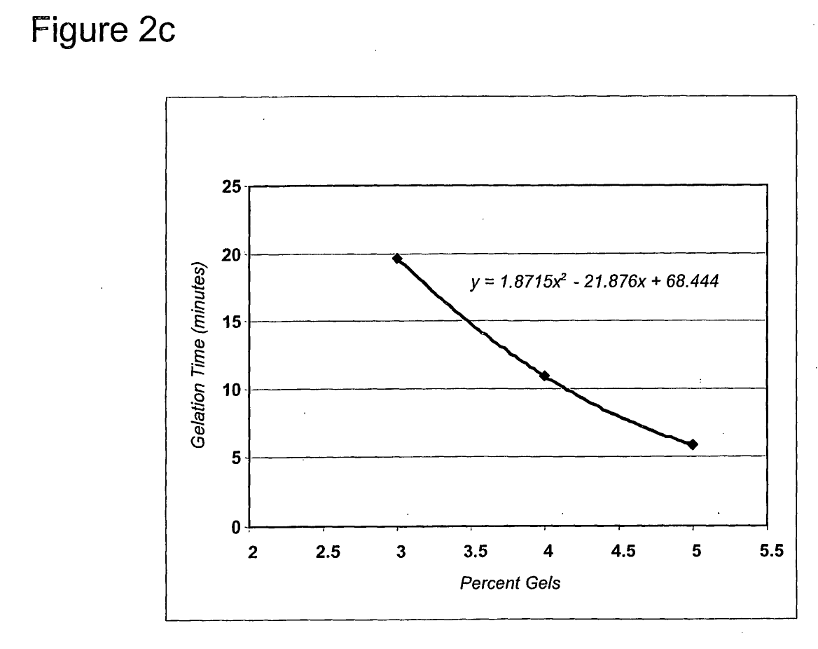

Gel permeation chromatography of formulation A (2% w/v P4-SH/P4-AM) was performed to compare the size of unreacted components with that of the network to demonstrate formation of a high molecular weight, branched molecule of molecular weight ˜1 million. FIG. 2A shows Gel permeation chromatograms of network formulation A (2% PEGs) and the individual PEG components (P4-SG and P4-AM)

Network Characterization: Determination of Kinetics of Branching and Gelation

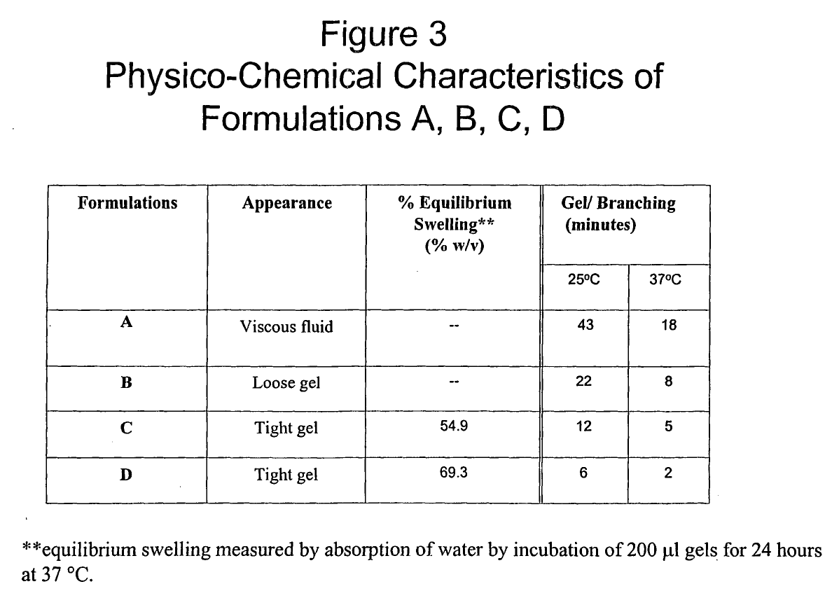

The time post-mixing to achieve maximum equilibrium branching or gel formation was determined by changes in shear viscosity measured by a DV-II Brookefield viscometer. The kinetics of hydrogel formation of formulations B-D were measured. The “onset of gelation”, characterized by rapid increases in solution viscosity, indicated the gel time. Data shown in FIG. 2B demonstrates gelation kinetics for formulations A (2% w/v P4-AM/P4 SG), B (3% w/v P4-AM/P4-SG), C (4% w/v P4-AM/P4-SG), and D (5% w/v P4-AM/P4-SG) measured by viscometry. As demonstrated in FIG. 2C, it is apparent that gels with a higher concentration of reacting polymers gelled faster than gels with a lower concentration of reacting polymer. As shown in FIG. 3, the crosslinking reaction was accelerated at higher temperatures for P4-AM/P4-SG formulations.

Network Characterization: Hardness or Softness of Gels

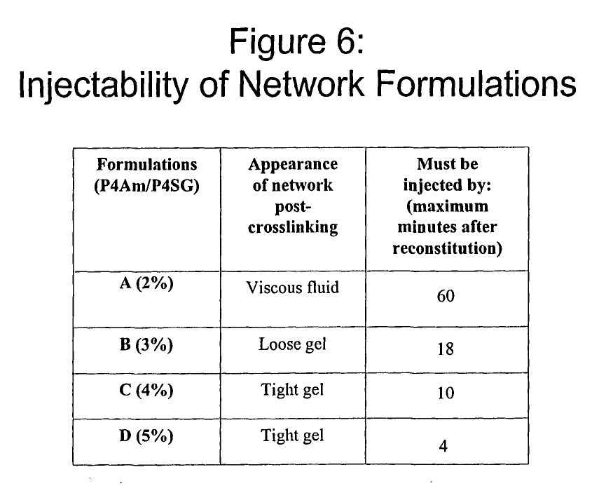

After gelation, the gels were removed from micro-centrifuge tubes and examined for texture, and physical attributes. In analytical chemistry terminology, the compression moduli σ (stressσ/strain) of networks (dynes/cm²) were determined to characterize crosslink densities, or “mesh size.” As demonstrated in the table in FIG. 6, a tightly crosslinked hydrogel formed from higher concentrations has a higher compression modulus than a loosely connected network formed when mixing components with lower concentrations, and is therefore characterized as “harder” (e.g., 5% w/v P4-AM/P4-SG gels were found to be “hard” whereas 3% w/v P4-AM/P4-SG gels were “soft”). Crosslink densities can control the ability of a molecule to diffuse through the network.

Compatibility of Plasmid DNA with Gel Forming Components

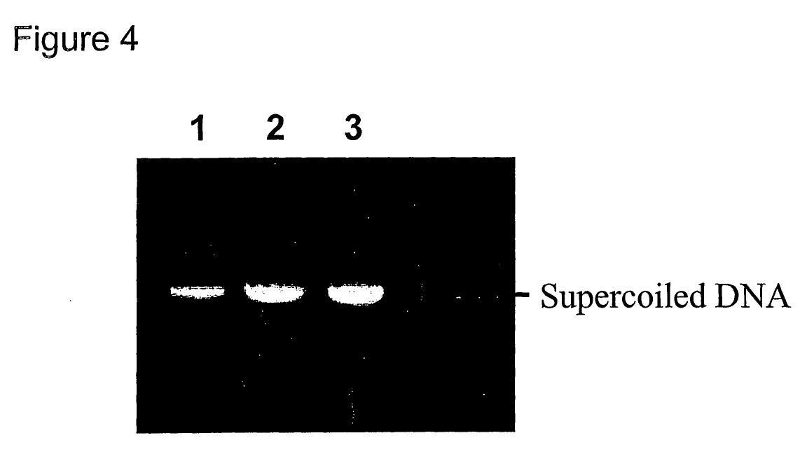

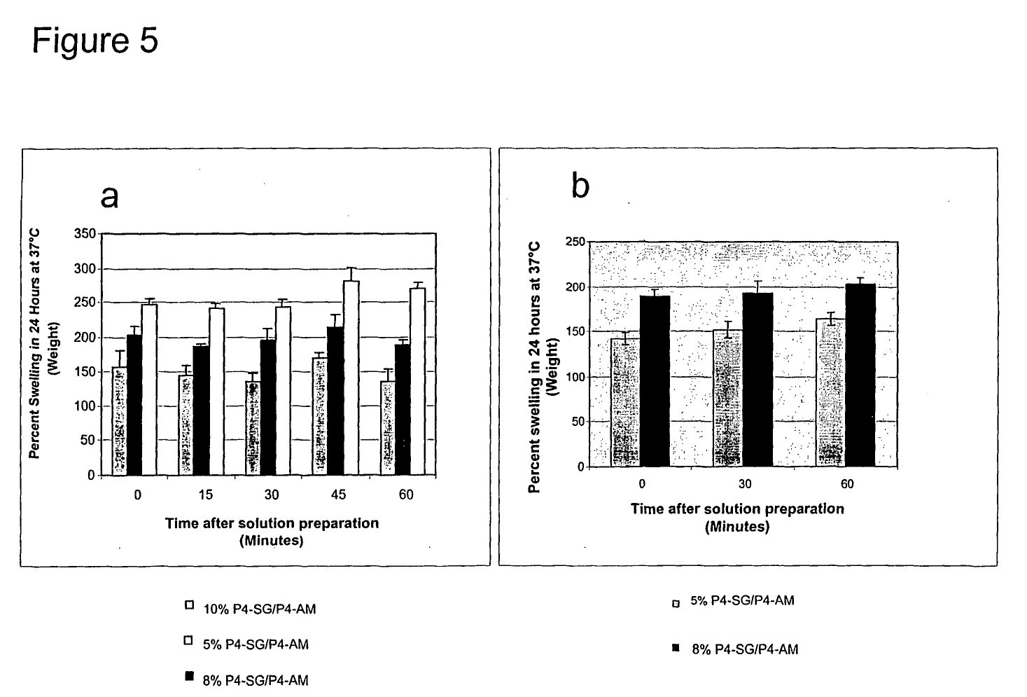

A compatibility experiment was performed to ensure that pre-mixing components 1 and 2 did not decrease the integrity (e.g., supercoiling) of plasmid DNA. Plasmid DNA (pDNA) (10 μg/ml) was mixed with either of the reacting polymers (2% w/v P4-AM or P4-SG, 5% w/v P4-AM or P4-SG) and incubated at room temperature for 30 minutes. FIG. 4 demonstrates percent supercoiling of the pDNA as subsequently determined by agarose gel electrophoresis. DNA supercoiling was found to not be affected by either of the non-gelled components. In another experiment, plasmid DNA was incorporated into 2% w/v and 3% w/v hydrogels, and then extracted into phosphate buffered saline to test if the supercoiling of the plasmid was compromised by the crosslinking reaction. The supercoiling of the network-extracted DNA was compared with control DNA that had not been incorporated into networks. No loss in DNA supercoiling was observed by incorporation of plasmid in networks. As shown in FIGS. 5A and 5B, the data demonstrates that DNA integrity was maintained in the presence of a cross-linked formulation.

Network Characterization: In Vitro Equilibrium Swelling

Equilibrium swelling can be used to characterize hydrogels. This method, when developed as a method of analysis, can be utilized effectively to determine reproducibility of a formulation. Networks containing plasmid DNA (with, without mPEG-DSPE) were prepared as described above except that the mixing was performed in a 96 well plate. Samples were incubated at 37° C. for 1 hour to allow complete gelation. The gels were removed from the wells and placed into scintillation vials. The vials were weighed, and 5 ml of Dulbecco's phosphate-buffered saline (PBS) was added. The vials were incubated overnight at 37° C. with gentle shaking. The buffer was then aspirated out of the vial and the gels were re-weighed. Equilibrium swelling was calculated as percentage increase in gel-weight using the following formula:

% Swelling=((Final weight of Gel)_{37° C., 24 hrs}−(Initial weight of Gel)_{37° C., 24 hrs}÷(Initial weight of Gel)_{37° C., 24 hrs}×100.

Measurements were performed on formulations prepared with different concentrations of the two reacting polymers, P4-AM and P4-SG, at different intervals following stock solution preparation. The graph in FIG. 5B demonstrates that percent swelling was unaffected for gels prepared at different intervals following solution preparation. The data in FIG. 5B demonstrate that it is evident that swelling increases with polymer concentration. Percent swelling is unaffected by the addition of nucleic acid (e.g., plasmid DNA at 1 mg/ml final concentration), or by the addition of components such as mPEG-DSPE. Thus, neither of these components is reactive with the gel components.

Network Characterization: In-Vitro Release of Plasmid DNA from P4-AM/P4-SG Gels

In-vitro release of DNA from hydrogels B-D was measured by incubation of plasmid-containing gels in phosphate buffered saline at 37° C. (200 μl hydrogel containing 200 μg of plasmid in a scintillation vial was incubated in 2 ml of PBS). At defined time points, the supernatant was removed and transferred to a new tube. An additional 2 ml of PBS was then added to each vial and the samples were returned to the incubator.

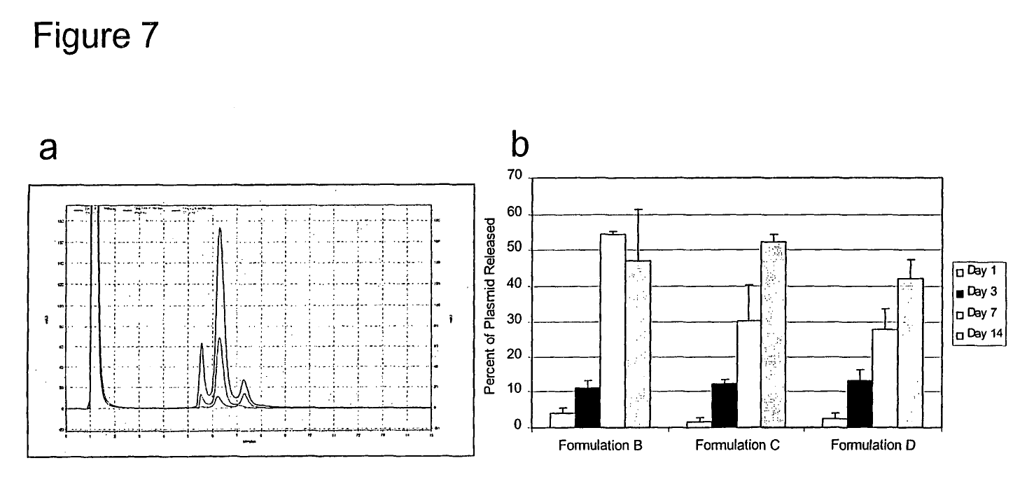

Percent DNA release from hydrogels was quantified using a DNA-NPR® (Tosoh-Biosep Inc.) anion exchange column using a gradient elution (HPLC Method: Buffer A: 0.56M sodium chloride in 50 mM Tris, pH 9.0; Buffer B: 1.2M sodium chloride in 50 mM Tris, pH 9.0; 0-30% Buffer B in 15 minute gradient elution). A standard curve was constructed with control unformulated DNA diluted in PBS at various concentrations and analyzed by HPLC. Relaxed and supercoiled plasmid peaks were identified in comparison with the retention time of the standards. FIG. 7A and 7B demonstrate in vitro release data. FIG. 7A shows a representative HPLC trace from DNA released from formulation C (4% w/v P4-AM/P4-SG); the second peak in the triplet set of peaks is supercoiled DNA. FIG. 7B shows cumulative release data from formulations B (3% w/v P4-AM/P4-SG), C (4% w/v P4-AM/P4-SG), and D (5% w/v P4-AM/P4-SG). The data indicate that plasmid can be released from the gels and that release is faster with gels containing a lower percentage of P4-AM and P4-SG.

Network Attributes: PEG-PEG Networks Protect Plasmid DNA from Serum Endonucleases

100 μl of cross-linked formulations (A=2% w/v P4-SG/P4-AM, B=3% w/v P4-SG/P4-AM) containing 30 μg of β-gal DNA, were incubated with shaking at 37° C. in 100 μl of a solution containing fresh BALB/c mouse serum in dilution ratios 1:40 to 1:80 for 30 minutes. Controls were incubated in serum-free buffer.

Endonuclease-based digestion of unformulated DNA and DNA in network formulations was compared by analysis of the plasmids on agarose gels.

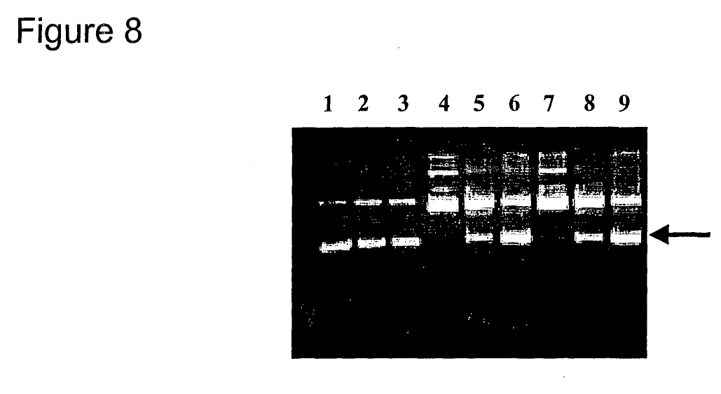

FIG. 8 demonstrates that both network formulations protected the plasmid DNA from serum endonucleases (lanes 5-6, 8-9), whereas unformulated DNA showed a loss of supercoiling after 30 minutes of incubation in both serum dilutions (lanes 4 and 7).

Network Characterization: Injectability

The injectability of the formulations was determined in the following manner:

P4-AM and P4-SG were loaded into separate 0.3 ml syringes, which were then joined via a syringe connector. Solutions of the components were mixed rapidly, and then retrieved into a single syringe. The mixed formulation was extruded through a 26 g needle at various time points post-mixing. The time within which a formulation could be injected was recorded in minutes. FIG. 6 demonstrates that formulations at higher concentrations gelled faster, lowering the time interval within which injection could occur.

Network Attributes: In Vivo In Situ Gel Formation in Muscle Tissue

To determine if the network formulations could be injected into the muscle of an animal and would form networks in situ in vivo, Evans Blue dye was added to formulations made up of 2%, 3%, 4%, 5%, 8%, or 10% w/v of total PEGs (P4-AM/P4-SG). The formulations were injected into the muscle tissue of mice.

Mice were sacrificed 60 minutes following injection, and the injected muscles were removed and examined for visible gels.

All formulations were injectable. Formulations containing 3%, 4%, 5%, 8%, and 10% w/v total PEGs (P4-AM+P4-SG) each formed transparent visible gels in the muscle tissue. The 2% w/v PEG formulation diffused throughout the muscle tissue as a viscous liquid.

Network Characterization: In-vivo Biodegradation

To study the biodegradation of the networks, 100 μl of formulation B (3% P4-SG/P4-AM) was injected per mouse, 50 μl per anterior tibialis muscle, two mice/group. Network-containing muscle tissue was excised at predetermined time points and the muscle was digested using the tissue digestion method described below.

Materials for Enzyme Digestion Method

Cysteine-HCl (99.9% purified, Sigma)

EDTA (Sigma)

50% Papain solution, 99.9% purified (Sigma)

Collagenase (98% purified, Sigma)

Calcium Chloride, 10% w/w in deionized water.

Enzyme Digestion Method

0.0606 g of cysteine-HCl and 2.915 g EDTA were weighed and added to a 100 ml volumetric flask, then filled to the 100 ml mark. The pH of the solution was adjusted to 6.25. The solution was bubbled with an inert gas such as argon to remove oxygen, and then stored at−20° C. until use.

5 ml of the 50% papain solution (Sigma) was pipetted in a 10 ml volumetric flask, then filled to the 10 ml mark to 10 ml with the cysteine/EDTA buffer. The solution was stored at−20° C. until use.

Collagenase (1.5 mg/ml) was prepared by weighing out 1.5 mg of collagenase and resuspending in 1 ml of cysteine/EDTA buffer.

1125 μl of the papain solution was added to a 15 ml centrifuge tube containing the tissue explant (weight of the explant should be between 30 and 600 mgs). 1125 μl of the collagenase solution and 750 μl of 10% calcium chloride was added to the centrifuge tube and mixed. The centrifuge tube containing the explant and the digestion cocktail was equilibrated for 8-12 hours in a water bath maintained at 37° C. This step resulted in the digestion of the tissue. After digestion, the pH of the digested dispersion was adjusted to 11.5 with 30 μl of 50% NaOH, and then the tube was placed in the 37° C. bath for an additional 8-12 hours. This step resulted in the digestion of the crosslinked network to the corresponding tetrameric PEG components. The pH of the solution was then adjusted back to 9 with aqueous HCl. The tissue debris was centrifuged for 0.5 hour, and then filtered through a 0.22 μm filter to prepare it for GPC analysis of PEG.

Percent PEG remaining at the tissue site over time was analyzed by gel permeation chromatography (Tosoh-Biosep TSK G3000PWXL column; mobile phase: 20 mM sodium monobasic phosphate buffer, pH 7.4).

The rate of in-vivo bioabsorption of these polyethylene glycol-based networks was determined by quantification of total PEGs remaining at the injected muscle site over time. The results demonstrate that ˜40% of total injected PEGs for formulation B had cleared from the site 33 days post-injection, and that 60-80% of the total injected PEG polymer was lost approximately 90 days post injection. The study demonstrated that the network delivery systems can be used in in vivo applications.

### Example 2

### Poly(ethylene oxide)-Poly(amidoamine) Networks via Formation of Amide Linkages

Materials

Poly(amidoamine), (PAMAM), Generation 0 (G0), 4 amine groups (Dendritech)

Poly(amidoamine), (PAMAM), Generation 1 (G1), 8 amine groups (Dendritech)

Poly(ethylene oxide)-tetrasuccinimidyl glutarate (P4-SG) (SunBio Systems)

Formulation

10% P4-SG (100 mg in 1 ml) was prepared in milliQ water and stored on ice. Equimolar concentrations of poly(amidoamine) G0 (10 mM or 0.5%) and G1 (10 mM or 0.5%) solutions were prepared by diluting the respective stock solutions in phosphate buffer (pH 8.0). The P4-SG solution, when mixed in equal volumes with either of the G0 or G1 solutions, formed a transparent soft gel. Gels with different crosslinking density could be formed by varying the concentrations of P4-SG and poly(amidoamine) G0 or G1. Different compositions (2%, 5%, 10%) containing PEG-DSPE and nucleic acid were also formulated, and all were found to be injectable.

Network Characterization: Determination of In Vitro Equilibrium Swelling

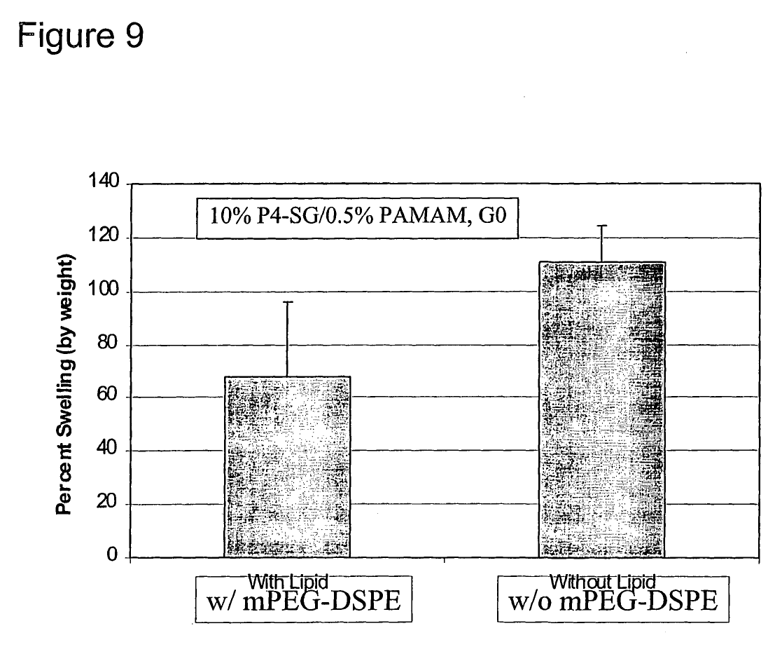

To characterize the PAMAM/P4-SG hydrogels, equilibrium swelling studies were performed. FIG. 9 demonstrates that the amount of swelling is much lower for these gels than for the PEG hydrogels, and the addition of lipid was found to decrease the swelling.

### Example 3

### Poly(ethylene oxide)-poly(ethyleneimine) Networks via Formation of Amide Linkages

Materials

Polyethyleneimine (PEI), 25 kD (Aldrich, Milwaukee, Wis., USA)

Poly(ethylene oxide)-tetrasuccinimidyl glutarate (P4-SG) (SunBio Systems)

Formulation Method

In situ crosslinking gels were formulated using poly(ethylenimine) and P4-SG. A 10% w/v solution of P4-SG was prepared in milliQ water. 100 mM of PEI (0.15% w/v) was prepared in phosphate buffer, pH 8.0. 100 μl of this solution (10 times molar excess) was added to the P4-SG solution and quick gelation was observed (<1 minute).

Gelation time can be controlled by altering the PEI or P4-SG concentration, and/or the pH of the solutions in which the individual polymers are resuspended. For example, a formulation containing 0.075% w/v PEI and 10% w/v P4-SG reconstituted at pH 8.0 has a gel time of 6 minutes at 25° C.

### Example 4

### Poly(ethylene oxide)poly(ethylene oxide) Networks (P4-SG/P4-SH) via Formation of Thioester Linkages

Materials

Poly(ethylene oxide)-tetrasuccinimidyl glutarate (P4-SG) (SunBio Systems)

Polyethylene oxide tetrasulfydryl (P4-SH) (SunBio Systems)

Method

100 μl of a solution of P4-SG (3% w/v) in milliQ water was mixed with 100 μl of a solution of 3% w/v P4-SH in phosphate buffer, pH 8.0 in a 1.5 ml centrifuge tube and incubated at 37° C. to form a 3% w/v hydrogel.

Similarly, gels of 4%, 10%, 20% and 30% w/v were also formed by mixing equal volumes of equimolar solutions of the two network forming polymers. After 30 minutes, the gels were retrieved and examined for their attributes.

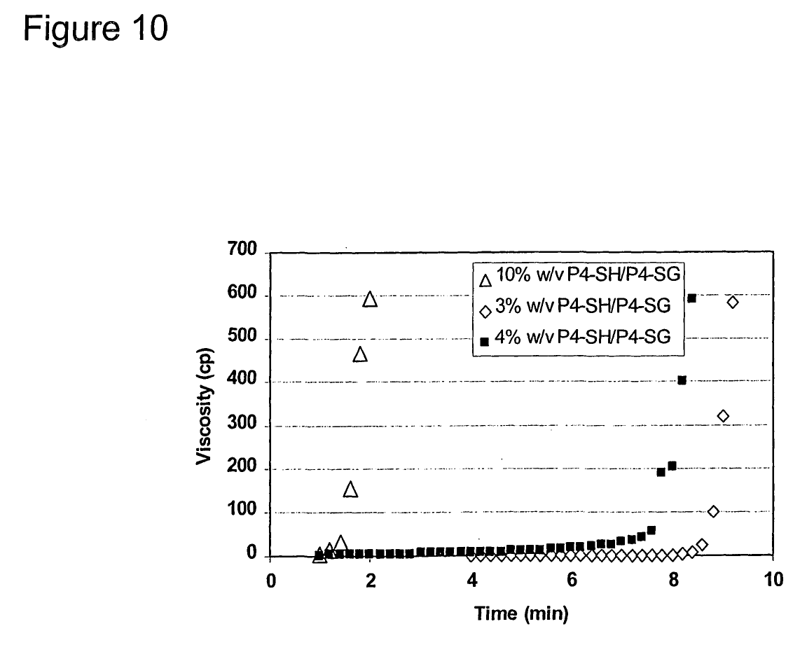

The 3% w/v gels were found to be soft, whereas the 4-30% w/v gels were “hard” and tightly crosslinked. Gel times for the 3%, 4%, and 10% w/v P4-SG/P4-SH formulations were measured in a Brookefield viscometer at temperatures of 25° C. and 37° C. As shown in FIG. 10, gel times accelerated with increased polymer concentrations.

### Example 5

### Modulation of Gene Expression in Murine Muscle via Modulation of Network Density

Use of a plasmid that encodes a secreted protein permits serum sampling and analysis for expressed protein without sacrificing the animal. For example, plasmids encoding secreted embryonic alkaline phosphatase gene, Factor VIII, Factor IX, erythropoetin (EPO), endostatin, various cytokines, insulin, and bone morphogenic protein (BMP) have been used for this purpose. A plasmid encoding the human secreted embryonic alkaline phosphatase gene (pgWiz™ SEAP, henceforth referred as “SEAP”) was used to monitor systemic expression. SEAP, a secreted form of the membrane bound placental alkaline phosphatase, has a half-life of from minutes to a few days in serum. A protein with a short half-life is especially useful to reliably determine expression kinetics.

Materials

pgWiz-SEAP, (Gene Therapy Systems Inc., San Diego, Calif., USA).

Polyethylene oxide-tetraamine (P4-AM) (SunBio Systems)

Poly(ethylene oxide)-tetrasuccinimidyl glutarate (P4-SG) (SunBio Systems)

mPEG-DSPE (Genzyme).

5-6 week old female C57B16 mice (Jackson Laboratories, Bar Harbor, Me., USA)

5-6 week old DBA/2 and Rag2 mice (Taconic, Germantown, N.Y., USA).

Formulations

DNA was amplified and purified using a Qiagen Endo-free™ kit (Qiagen Inc., Valencia, Calif., USA) or was purchased from Aldevron LLC (Fargo, N.Dak., USA).

All formulations were prepared by mixing of two solutions, one containing a pre-weighed amount of P4-AM dissolved in 0.1M potassium phosphate buffer, pH 8.0, and the other containing an equimolar amount of P4-SG dissolved in cold deionized water containing SEAP plasmid DNA (100 μg/100 μl final volume of formulation) and mPEG-DSPE (10 μg/100 g final volume of formulation).

Formulation A included 2% w/v P4-SG/P4-AM cross-linked into a viscous branched polymer.

Formulations B-D (3, 4, and 5% w/v P4-SG/P4-AM, respectively) included equimolar amounts of the same components as A, but at higher concentrations. These formulations cross-linked into tissue-conforming hydrogels in situ, post-injection into muscle.

The solutions were freshly prepared and injected into mouse muscle immediately after mixing all formulation components.

Animal Experiments

Mice were mildly anesthetized using isofluorane and injected with different cross-linked network formulations or with unformulated plasmid DNA (in saline) bilaterally into the anterior tibialis muscles. All animals were injected with 100 μg of plasmid DNA in an injection volume of 50 μl per muscle.

At different timepoints post-injection, mice were anesthetized and blood was collected retro-orbitally. Serum was separated from red blood cells by centrifugation and stored at −80° C. until assays were performed.

SEAP Assay

Levels of enzymatically active SEAP in mouse serum were measured using the Tropix Phospha-Light® luminometric assay kit (Applied Biosystems, Foster City, Calif., USA). Assays were performed according to the manufacturer's protocol except that samples for the standard curve were prepared in normal mouse sera (Stellar Biosystems, Columbia, Md., USA) diluted 1:4.

All experimental serum samples were also diluted 1:4 in manufacturer-supplied dilution buffer.

Luminescence measurements were performed using a Topcount® plate reader (Packard Instruments, Illinois) following 40 minutes of incubation in the reaction buffer. Serum SEAP levels at each timepoint were expressed in nanograms/ml using the standard curve generated from the positive control (purified human placental alkaline phosphatase) supplied with the assay kit. The data were further analyzed using a Thompson-Tau outlier analysis as described in Wheeler and Ganji, “Introduction to Engineering Experimentation,” Prentice Hall, pp. 142-145 (1996) and plotted as averages and standard deviations.

Results

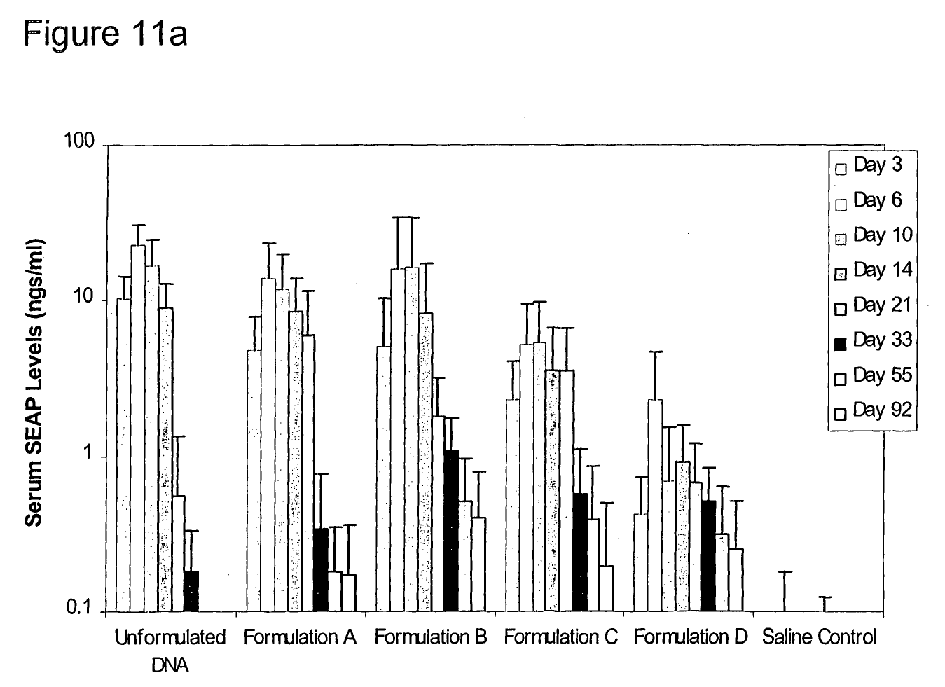

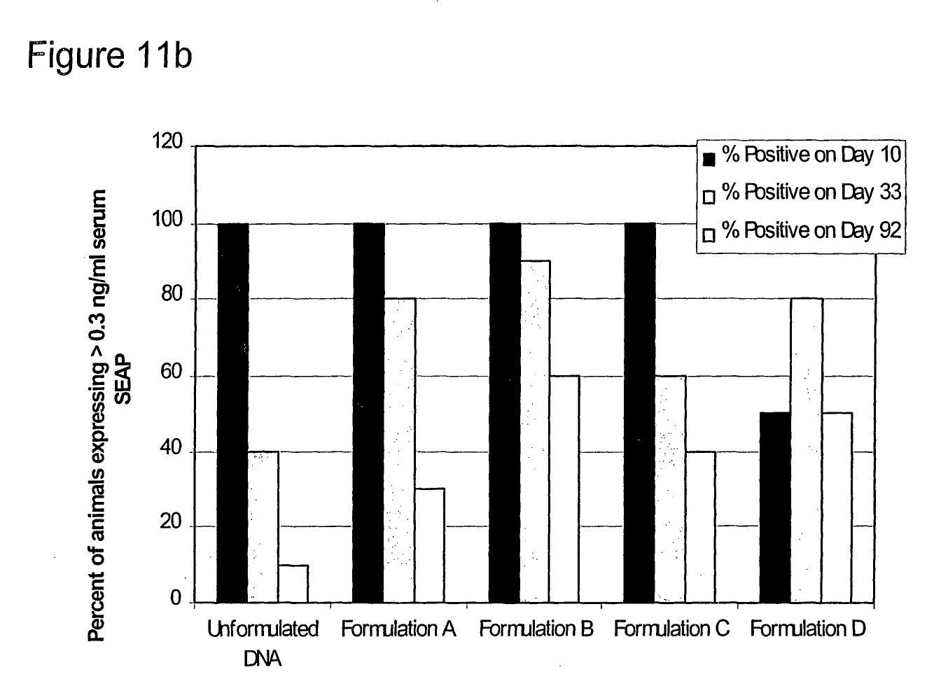

Administration of each of the network formulations resulted in detectable levels of serum SEAP for extended periods of time. FIG. 11A shows that all networks with higher crosslink densities (i.e., formulations C and D) produced significant serum levels of SEAP expression compared to lightly crosslinked networks (i.e., formulations A and B). To evaluate the long-term expression of DNA released from the network formulations, percent positive animals (as measured by animals expressing more than 300 pg/ml of serum SEAP, a level that is 2-3 fold higher than background serum SEAP levels in saline injected mice) were plotted for each formulation FIG. 11B demonstrates that DNA delivery from the networks resulted in long-term expression of the encoded protein in serum, whereas protein levels in animals injected with unformulated DNA dropped precipitously after 3-4 weeks.

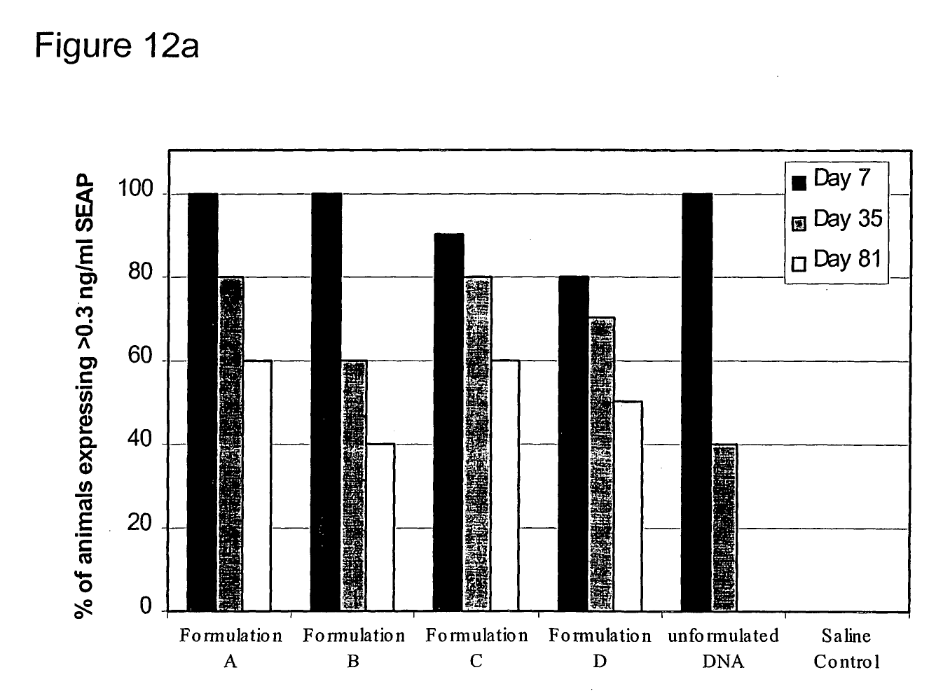

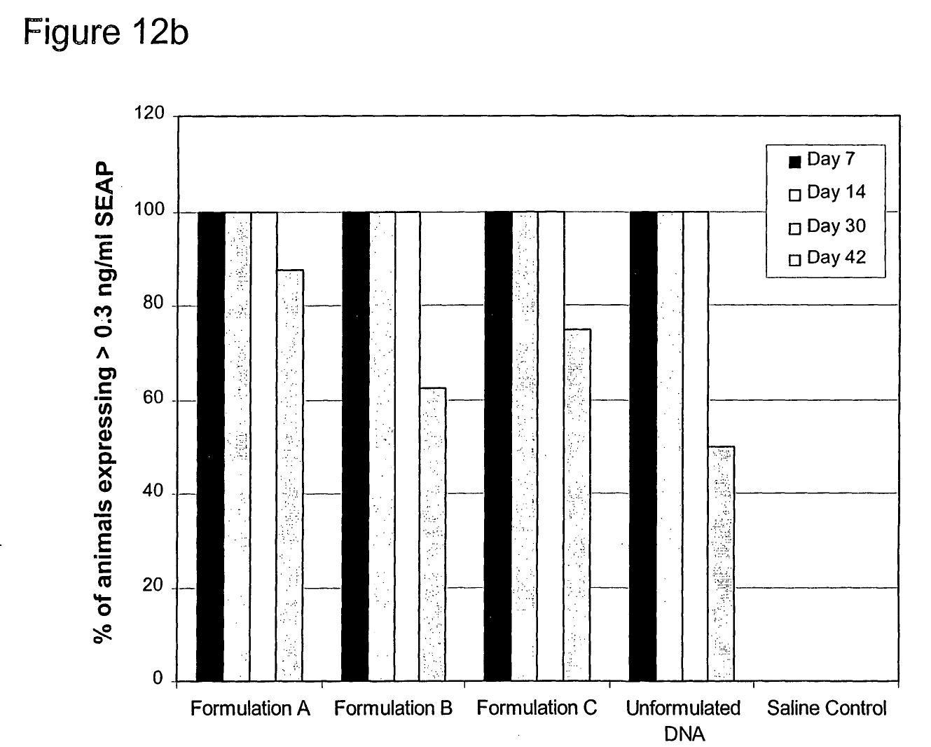

One hypothesis for transient expression of proteins following intramuscular injections of plasmids is antibody-directed complement-mediated cytotoxicity (ADCC). To evaluate if the sustained protein expression kinetics observed in immunocompetent animals was apparent in complement deficient mice incapable of mounting ADCC, DBA/2J mice, deficient in a component of complement, were injected with unformulated DNA or DNA in network formulations. FIG. 12A show that in complement-deficient animals, network injection produced more sustained expression of SEAP compared to that produced by unformulated DNA.

To further evaluate the effect of long-term protein expression in immunodeficient animals, RAG2 knock out mice, incapable of V(D)J recombination, and thus lacking mature B and T cells, were administered pSEAP plasmid as unformulated DNA or in network formulations. FIG. 12B shows that serum SEAP levels from animals injected with network associated DNA were sustained longer than those from the groups injected with unformulated DNA.

### Example 6

### Delivery of Nucleic Acid in P4-AM/P4-SG Networks Followed by Electroporation Enhances Gene Expression

Materials

Polyethylene oxide-tetraamine (P4-AM) (SunBio Systems)

Poly(ethylene oxide)-tetrasuccinimidyl glutarate (P4-SG) (SunBio Systems)

mPEG-DSPE (Genzyme)

SEAP plasmid DNA (Gene Therapy Systems)

5-6 week old female C57B16 mice (Jackson Laboratories)

Formulations

3% w/v P4-AM/P4-SG was formulated with mPEG-DSPE (10 μg/100 μl) and (100 μg/100 μl) SEAP DNA. The gel was identified as a GT20 gel. GT20 denotes a gel time of 20 minutes post reconstitution with buffer at pH 8 as measured by viscometry at 25° C.

Method

Mice were mildly anesthetized using isofluorane and injected with different crosslinked network formulations or with unformulated plasmid DNA (in saline) bilaterally into the anterior tibialis muscles. All animals were injected with 100 μg of plasmid DNA in an injection volume of 50 μl per muscle.

The mouse muscles were electroporated immediately post-injection of the formulations with 200 V/cm, 8 pulses, 20 ms pulse width at 1 second intervals (Genetronics electroporator, ECM 830; BTX Inc., San Diego, Calif., USA).

Serum collection, SEAP assays, and data analysis using Thompson-Tau Outlier analysis were performed as in example 5.

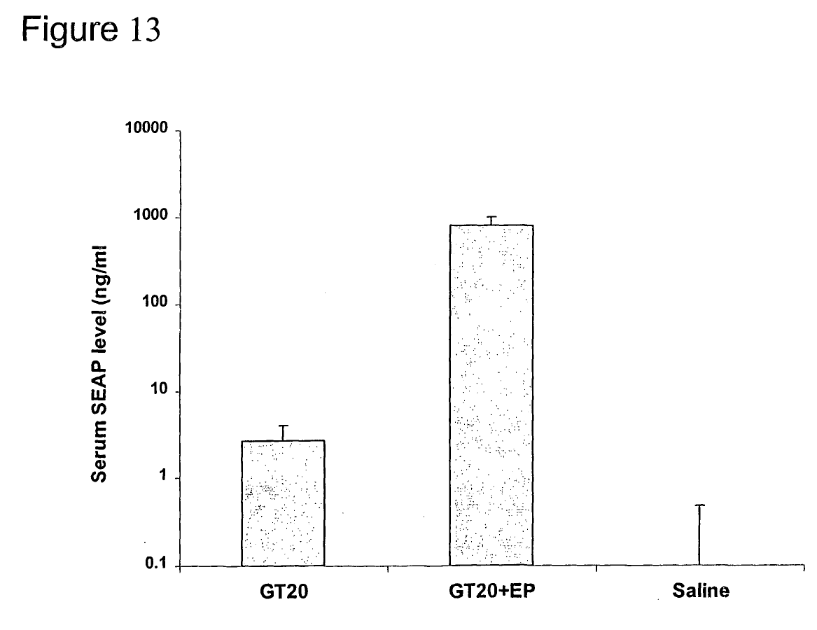

The data shown in FIG. 13 demonstrates enhancement of SEAP expression in network formulation by electroporation.

### Example 7

### Addition of Excipients to P4-AM/P4-SG Networks Enhances Gene Expression

Materials

Polyethylene oxide-tetraamine (P4-AM) (SunBio Systems)

Poly(ethylene oxide)-tetrasuccinimidyl glutarate (P4-SG) (SunBio Systems)

mPEG-DSPE (Genzyme)

SEAP plasmid DNA (Gene Therapy Systems)

5-6 week old female C57B16 mice (Jackson Laboratories)

Formulations

3% w/v P4-AM/P4-SG was formulated with mPEG-DSPE (10 μg/100 μl) and (100 μg/100 μl) SEAP DNA. The gel was also identified as a GT20 gel. Various excipients were added to the DNA-containing P4-SG solution, before mixing with the P4-AM solution. The final concentrations of these excipients in the formulation were: sodium lauryl sulfate (SDS, 0.1% w/v)(Sigma), pluronic L62 (0.1% w/v)(BASF) Magainin I (0.025% w/v) (Sigma), and poly(amidoamine) (PAMAM; Dentritech) G0 (0.15% w/v). More specifically, sodium lauryl sulfate is classified as a anionic lipid, pluronic L62 as a non-ionic surfactant, Magainin I as a cationic peptide, and PAMAM G0 as a cationic 4-armed polymer.

Method

Mice were mildly anesthetized using isofluorane and injected with different crosslinked network formulations or with unformulated plasmid DNA (in saline) bilaterally into the anterior tibialis muscles. All animals were injected with 100 μg of plasmid DNA in an injection volume of 50 μl per muscle (n=8 per group).

Serum collection by retro-orbital bleeding, SEAP assays, and data analysis using Thompson-Tau Outlier analysis were performed as in example 5.

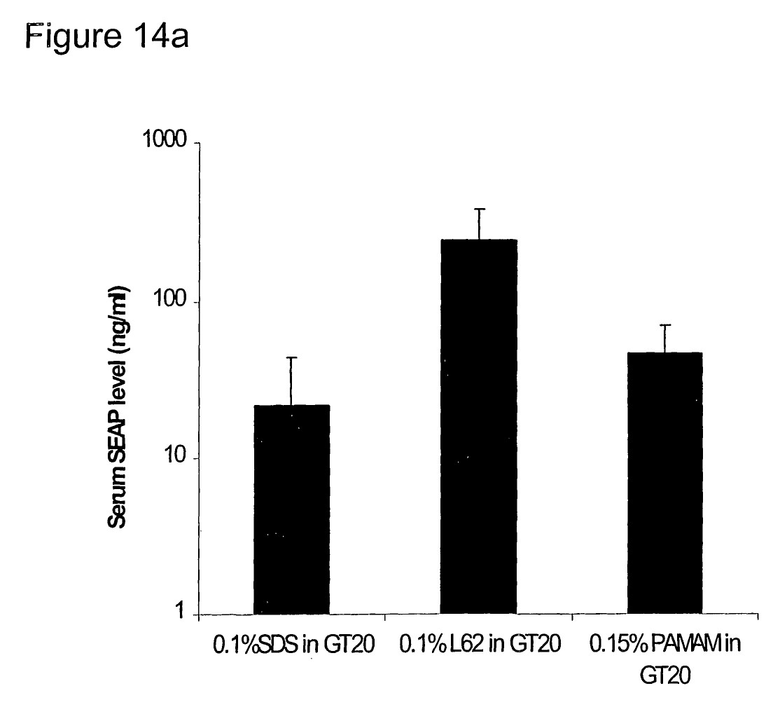

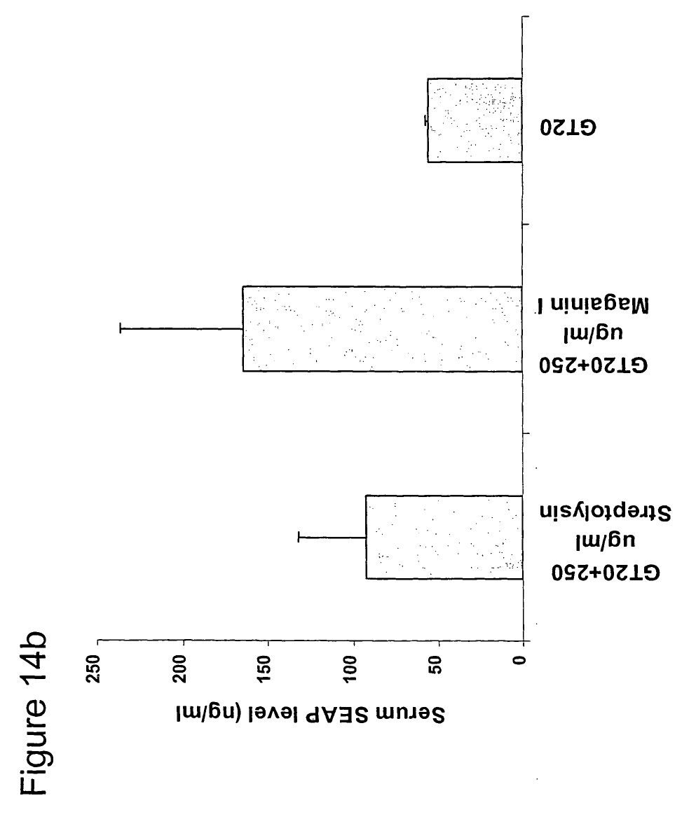

FIGS. 14A and 14B demonstrate that SEAP expression was found to be enhanced by the addition of these excipients to the network formulations.

### Example 8

### Gene Expression in Mouse Muscle Induced by P4-SG/P4-SH Networks

Materials

Poly(ethylene oxide)-tetrasulfydryl (P4-SH) (SunBio Systems)

Poly(ethylene oxide)-tetrasuccinimidyl glutarate (P4-SG) (SunBio Systems)

mPEG-DSPE (Genzyme)

SEAP plasmid DNA (Gene Therapy Systems)

5-6 week old female C57B16 mice (Jackson Laboratories)

Formulations

DNA was amplified and purified using a Qiagen Endo-free® kit (Qiagen Inc., Valencia, Calif.) or was purchased from Aldevron LLC (Fargo, N.Dak.).

All formulations were prepared by mixing of two solutions, one containing a pre-weighed amount of P4-SH dissolved in 0.1M potassium phosphate buffer, pH 8.0, and the other containing an equimolar amount of P4-SG dissolved in cold deionized water containing SEAP plasmid DNA (100 μg/100 μl final volume of formulation) and mPEG-DSPE (10 μg/100 μl final volume of formulation).

Two formulations were tested: Formulation A: 3.5% w/v of each P4-SH/P4-SG gelled after 20 minutes at 25° C.; and Formulation B: 5% w/v of each P4-SH/P4-SG gelled after 10 minutes at 25° C. The solutions were freshly prepared and injected into mouse muscle immediately after mixing all of the formulation components.

Animal Experiments

Mice were mildly anesthetized using isofluorane and injected with different crosslinked network formulations or with unformulated plasmid DNA (in saline) bilaterally into the anterior tibialis muscles. All animals were injected with 100 μg of plasmid DNA in an injection volume of 50 μl per muscle. There were 8 animals per group.

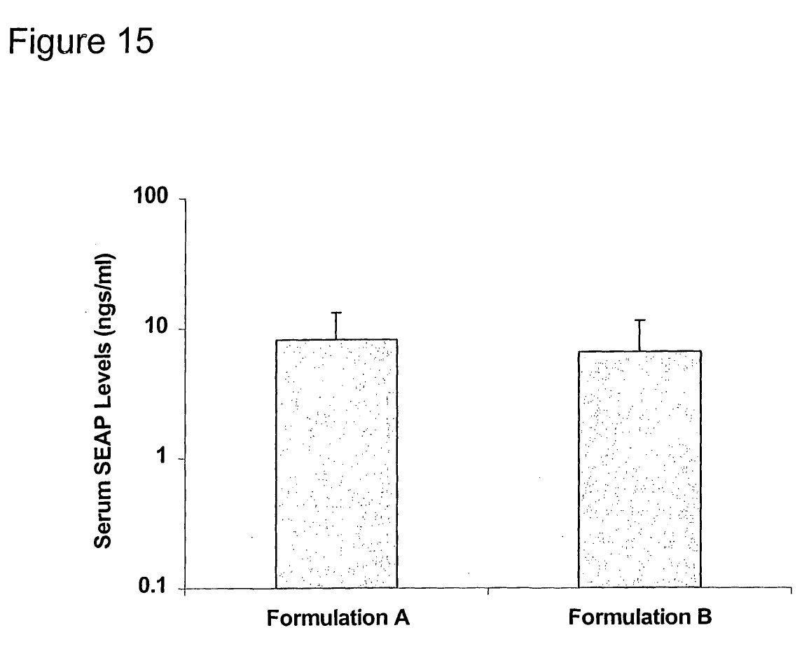

At day 7 post-injection, mice were anesthetized blood was collected, serum prepared and analyzed as in Example 5. As shown in FIG. 15, formulations A and B both induced high levels of gene expression in mice.

### Example 9

### Network-Mediated (P4AM-P4-SG) Gene Expression in Mouse Mucosa

Materials

pgWiz-SEAP, (Gene Therapy Systems Inc., San Diego, Calif.).

Poly(ethylene oxide)-tetraamine (P4-AM) (SunBio Systems)

Poly(ethylene oxide)-tetrasuccinimidyl glutarate (P4-SG) (SunBio Systems)

mPEG-DSPE (Genzyme)

5-6 week old female C57B16 mice (Jackson Laboratories)

Methods

DNA was amplified and purified using a Qiagen Endo-free® kit (Qiagen Inc., Valencia, Calif., USA) or was purchased from Aldevron LLC (Fargo, N.Dak., USA).

All formulations were prepared by mixing of two solutions, one containing a pre-weighed amount of P4-AM dissolved in 0.1M potassium phosphate buffer, pH 8.0, and the other containing an equimolar amount of P4-SG dissolved in cold deionized water containing SEAP plasmid DNA (100 μg/100 μl final volume of formulation) and mPEG-DSPE (10 μg/100 μl final volume of formulation).

GT5: 5% w/v of each of P4-AM and P4-SG gelled after 5 minutes at 25° C. The solutions were freshly prepared and injected into mouse rectum immediately post mixing, of all formulation components.

Animal Experiments

Mice were mildly anesthetized using isofluorane and injected with different crosslinked network formulations or with unformulated plasmid DNA (in saline) into the rectum 3.5 cm from the anus. All animals were injected with 100 μg of plasmid DNA in an injection volume of 50 μl. There were 5 animals per group.

At day 8 post-injection, mice were anesthetized blood was collected, serum prepared and analyzed as in example 5. Animals receiving unformulated DNA did not show SEAP expression. GT5 formulations induced significant levels of gene expression in 3 of 5 mice.

### Example 10

### Demonstration of Immune Responses to DNA Encoded Antigen Following IM Injections with Plasmid in P4-AM/P4-SG Networks

Materials

Poly(ethylene oxide)-tetraamine (P4-AM) (SunBio Systems)

Poly(ethylene oxide)-tetrasuccinimidyl glutarate (P4-SG) (SunBio Systems)

mPEG-DSPE (Genzyme)

The synthetic peptide, TPHPARIGL, representing the naturally processed H-2 L^{d} restricted T cell epitope spanning amino acids 876-884 of β-gal and IPQSLDSWWTSL, the H-2 L^{d} epitope corresponding to residues S28-39 of hepatitis B surface Ag (HBsAg), were synthesized by Multiple Peptide Systems (San Diego, Calif.) to a purity of >90% as assessed by reverse phase high-pressure liquid chromatography (RP-HPLC). The identity of each peptide was confirmed by mass spectrometry.

pCMV/β-gal encoding Escherichia coli β-gal driven by the human CMV intermediate early promoter was used as the reporter gene for all immunizations.

BALB/c mice, 6-10 wk of age

CT26.WT and CT26.CL25 cell lines. CT26.WT is a clone of CT26, a BALB/c (H-2^{d}) undifferentiated colon adenocarcinoma. CT26.CL25 is a CT26.WT clone stably transfected with the lacZ gene. Cell lines were maintained in RPMI 1640, 10% heat-inactivated fetal calf serum (FCS; Life Technologies, Grand Island, N.Y.), 2 mM L-glutamine, 100 μg/ml streptomycin, and 100 U/ml penicillin (Life Technologies, Grand Island, N.Y.). CT26.CL25 was maintained in the presence of 400 μg/ml G418 sulfate (Life Technologies, Grand Island, N.Y.).

Formulations

All formulations were prepared by mixing of two solutions, one containing a pre-weighed amount of P4-AM dissolved in 0.1M potassium phosphate buffer, pH 8.0, and the other containing a pre-weighed amount of P4-SG dissolved in cold deionized water containing β-gal DNA (100 μg/100 μl of formulation) and mPEG-DSPE (10μg/100 μl of formulation). Formulation A included 2% w/v P4-AM/P4-SG and created a viscous branched polymeric network post-mixing of the components. Formulation B included 3% w/v P4-AM/P4-SG and formed a hydrogel post-mixing. The solutions were freshly prepared at room temperature, mixed and injected immediately.

Physico-chemical Characterization of the Formulations

The molecular weight and size distribution profile of formulation A was determined to be one million by aqueous gel permeation chromatography using a TSK Gel Mixed Bed column with 0.02M phosphate buffer, pH 7.5, as the mobile phase. The network had a fluid viscosity of ˜5 cp, as measured by Brookefield rheometry. The gel point of formulation B was 11 minutes at 37° C. as measured by Brookefield rheometry.

Immunizations

Mice were mildly anesthetized using isoflurane and injected with different crosslinked network formulations or saline bilaterally into the anterior tibialis muscles. All animals were injected a single time with 30 μg of plasmid DNA in an injection volume of 50 μl per muscle. In a separate experiment, dissection of the muscle site approximately an hour post injection of formulation B demonstrated presence of a hydrogel conformed to tissue. Examination of the muscle site an hour post-injection of formulation A demonstrated formation of a thick, viscous gelatinous material.

ELISA Assay

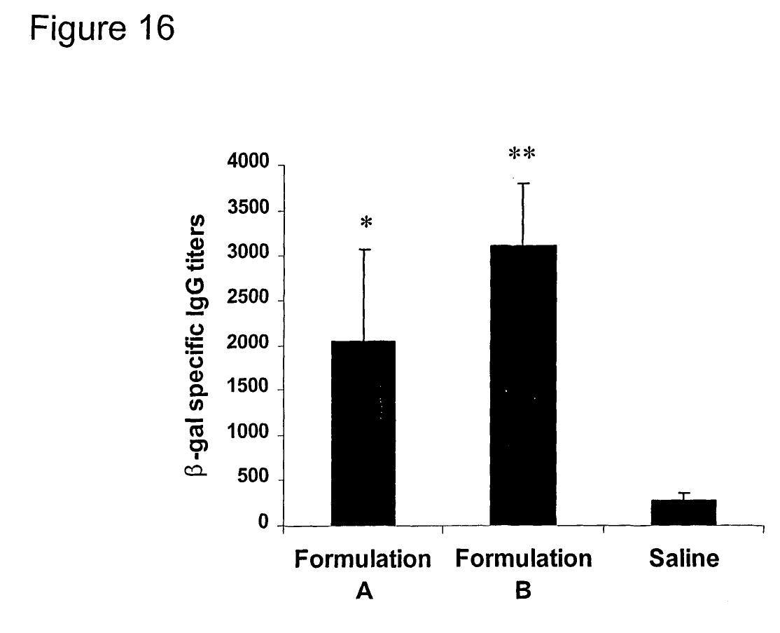

Sera was collected from mice by retro-orbital bleeding at 12 weeks post-immunization. Titers of β-gal specific antibodies at 12 weeks were measured by a standard ELISA protocol. β-gal titers were defined as the highest serum dilution that resulted in an absorbance (OD 405) value twice than that of non-immune sera at that dilution. FIG. 16 demonstrates that administration of DNA in networks derived from both formulations stimulated robust β-gal antibody responses measured 12 weeks post injection. Similar results were obtained in two separate experiments with identical formulation groups.

Proliferative T Cell Responses

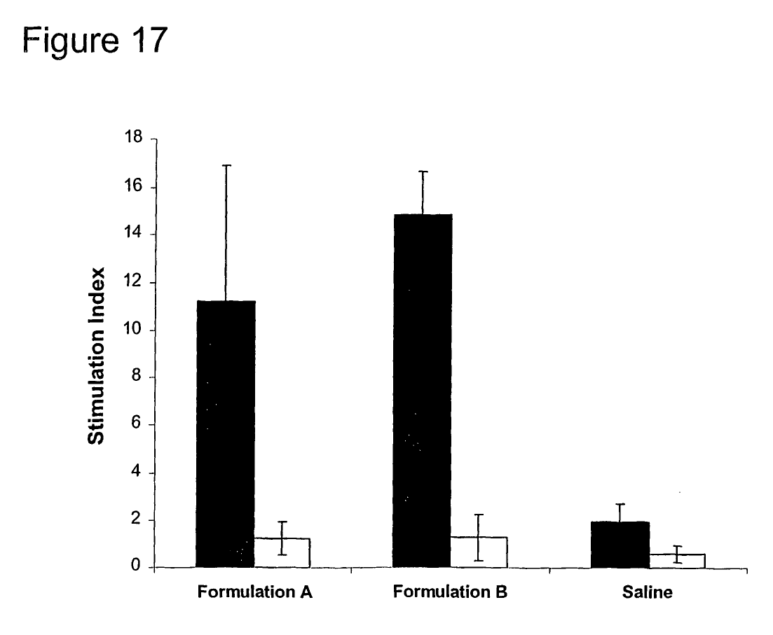

T cells from pooled (n=4) splenocytes of immunized or naive mice were purified using T cell enrichment columns according to the manufacturer's instructions (R&D Systems, Minneapolis, Minn.) at 12 weeks post-immunization. T cell proliferation assays were performed by incubating purified T cells and syngeneic irradiated splenocytes (2×10⁵ each) in the presence of 30 μg/ml of β-gal or chicken ovalbumin protein at 37° C. for 72 hrs. Cultures were pulsed with 1 μCi of tritiated thiamidine (³H-TdR) and incubated for 20 hours. Cells were then harvested and radioactivity measured on a beta counter. FIG. 17 shows that delivery of DNA in both network formulations induced β-gal specific proliferative T cell responses. This type of response is usually associated with a T helper restricted T cell population. Similar results were obtained in two separate experiments with identical formulation groups.

Gamma-Interferon ELISpot

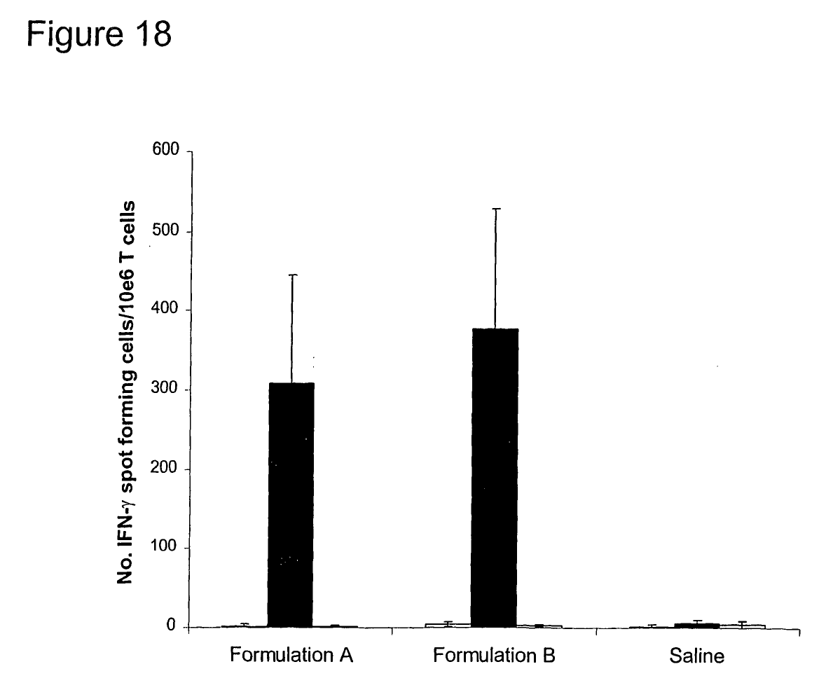

T cells from pooled (n=4) splenocytes of immunized or naive mice were purified using T cell enrichment columns according to the manufacturer's instructions (R&D Systems, Minneapolis, Minn.) at 12 weeks post-immunization. Purified T cells (2×10⁵) were stimulated with 2×10⁵ irradiated β-gal or HBV peptide pulsed syngeneic spleen cells for 24 hrs. The MHC Class I restricted T cell response elicited by these formulations was measured in a gamma-interferon (γ-IFN) enzyme-linked immunospot ELISpot) assay according to the manufacturer's directions (R&D Systems, Cat# EL485, Minneapolis, Minn., USA). Spots were enumerated electronically. FIG. 18 demonstrates that responses were detected at both the 12 week time points and were higher in mice given formulation A in comparison to those of mice receiving formulation B.

Tumor Protection Studies

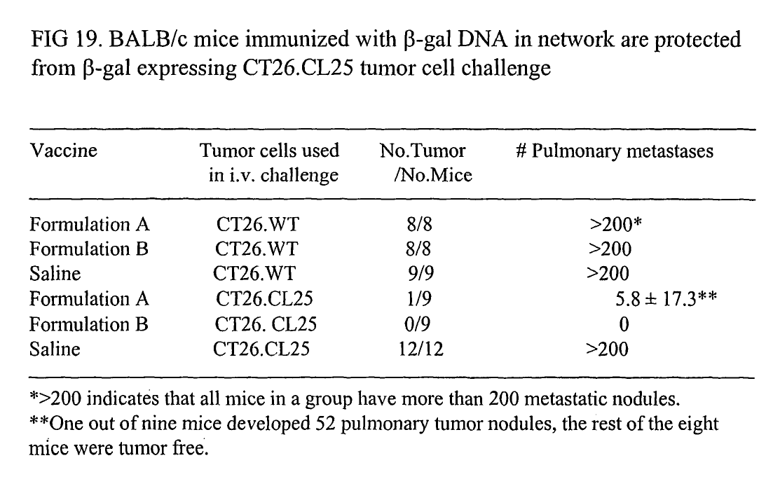

Mice were challenged intravenously with 5×10⁵ CT26.WT or CT25.CL25 cells post immunization with formulated DNA or saline control. Mice were sacrificed on day 13, lungs were isolated and stained with 0.2% X-gal solution after fixing with 0.25% glutaradehyde/0.01% formalin in PBS. Tumor nodules could then be visualized and enumerated. The protective response to this tumor is dependent on the class I restricted T cell response. Examination of lungs harvested on day 13 after tumor inoculation indicated the presence of multiple pulmonary metastases in all mice challenged with the CT26.WT cell line. Mice immunized with network entrapped DNA and challenged with the CT26 β-gal expressing tumor (CT26.CL25) were protected from metastases. As demonstrated by the data in the table of FIG. 19, all but one mouse had completely clear lungs.

### Example 11

### Preparation of A Lyophilized Formulation

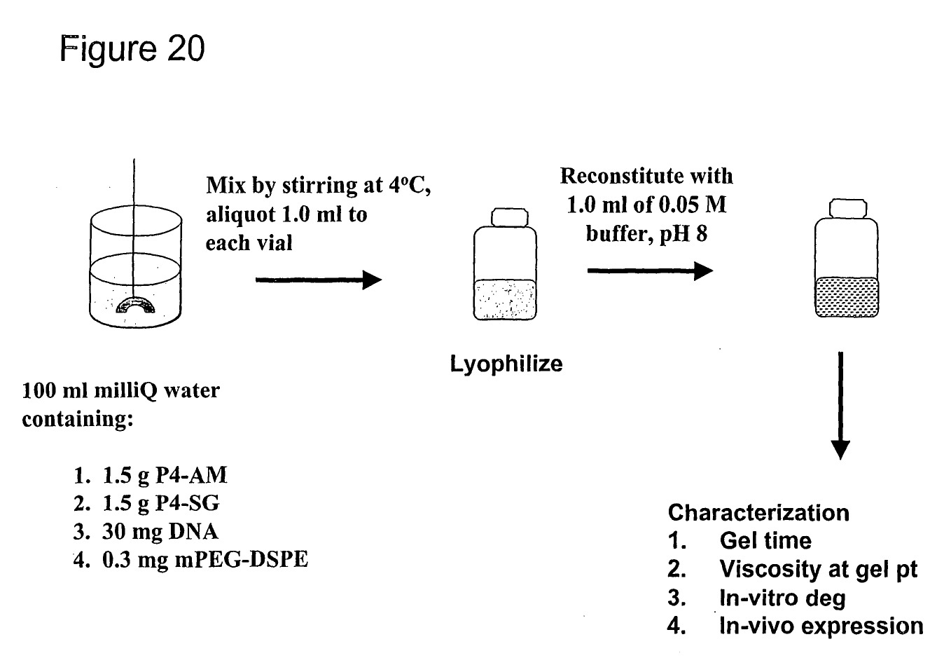

A schematic of a method for formulating a “one vial” lyophilized product that contains an excipient(s) such as a lipid, unreacted PEG-amine, unreacted PEG-succinimidyl glutarate, and a nucleic acid is provided in FIG. 20. At pHs greater than 7.0, the two PEG components mutually react to form a crosslinked network. Therefore, the pH of the solution containing the two PEG components was maintained below this threshold (e.g., the pH is maintained at 5.5 by the dissolution of the components in deionized water).

In this example, the reactivity of the two PEG components was also controlled by temperature. At 37° C., the gel-forming reaction proceeded at a faster rate than it did at 4° C. Therefore, the reaction in this example was maintained at approximately 0 to 4° C. (an ice water slurry).

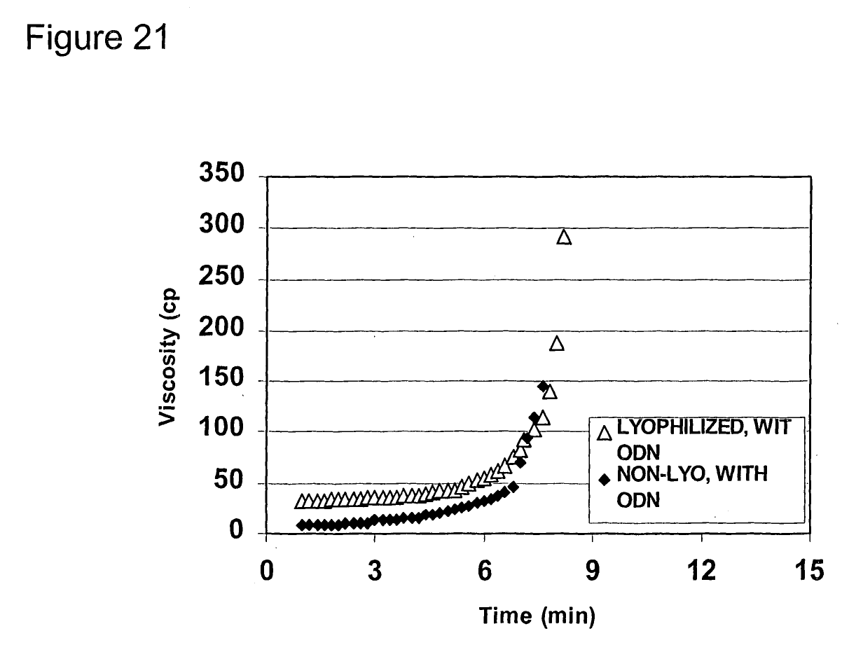

FIG. 21 shows a schematic for characterization of gels at lower temperature. After the mixing of the components, vials containing the DNA were filled with the solution and then lyophilized. The lyophile was reconstituted with phosphate buffered saline, pH 8.0, and gelation times (onset of gelation) were measured. A 3% w/v gel formed in approximately 25 minutes at 25° C. and did not vary from the gel time of a non-lyophilized formulation.

Lyophilization was also performed by mixing solutions of the reactive polymers (e.g., P4-SG and P4-SH), maintaining a pH of below 7, and lyophilizing in the absence of nucleic acid. In this instance, the nucleic acid was added to the formulation upon reconstitution. As shown in FIG. 21, gel times for formulations prepared in this way did not vary by the lyophilization procedure.

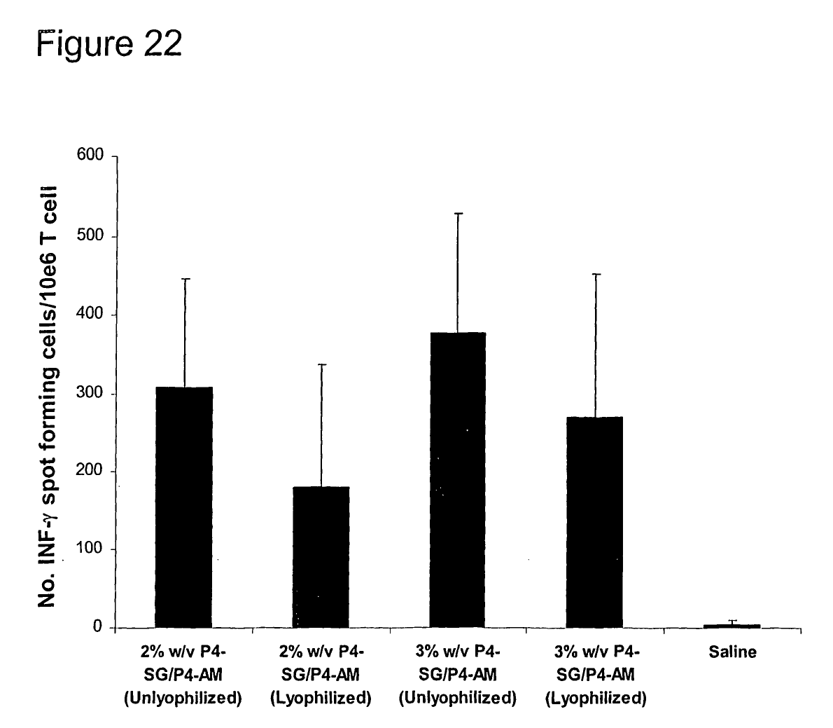

Solutions prepared from reconstituted vials were injected into mouse muscles within 5-7 minutes after reconstitution using the same DNA dose, immunization and assay protocols as described in Example 10. Formulations injected were 2% w/v P4-SG/P4-AM and 3% w/v P4-SG/P4-AM. The MHC Class I restricted T cell response elicited by these formulations was measured in a gamma-interferon (γ-IFN) ELISPOT assay according to the manufacturer's directions (R&D System, Minneapolis, Minn.). Spots were enumerated electronically. FIG. 22 shows that responses for both formulations were analyzed at 12 weeks post immunization. The results were statistically equivalent indicating that lyophilization does not adversely affect the ability of the formulation to function in vivo.

### Example 12

### Generation of Networks (P4-AM/P4-SG) Containing Oligonucleotides

Materials

Polyethylene oxide tetraamine (P4-AM) (SunBio Systems)

Poly(ethylene oxide)-tetrasuccinimidyl glutarate (P4-SG) (SunBio Systems)

Oligonucleotides with phosphorothioate or phosphodiester backbones (Oligos, etc., Wilsonville, Oreg., USA)

Methods

100 μl of a solution of P4-SG (5% w/v in milliQ water) (100 μg) was mixed with 100 μl of a 5% w/v P4-AM and oligophosphorothioate (10 μg/μl) (in phosphate buffer, pH 8.0) solution and incubated at 37° C. The onset of gelation was determined to be approximately 8 minutes at 37° C. by Brookefield rheometry and the formation of a soft gel was confirmed.

Formulations at concentrations of 5 and 10% w/v PEGs CP4-AM and P4-SG) with and without oligonucleotide were also prepared, and the formation of gels was noted in all cases.

In Vitro Release of Oligonucleotides

100 μl of a solution of P4-SG in milliQ water was mixed with 100 μl of a solution of P4-AM and 1 μg/μl of oligophosphorothioate (in phosphate buffer, pH 8.0) in a 1.5 ml centrifuge tube and incubated at 37° C. After 1 hour, the gel was retrieved and placed in a new centrifuge tube with 1 ml of phosphate buffered saline, pH 7.4. The gels were incubated at 37° C. At each timepoint, 800 μl of supernatant was retrieved and transferred to a new tube. To the tube containing the gel was added 800 μl of fresh buffer. The supernatant was analyzed for oligophosphorothioate content by anionic exchange chromatography.

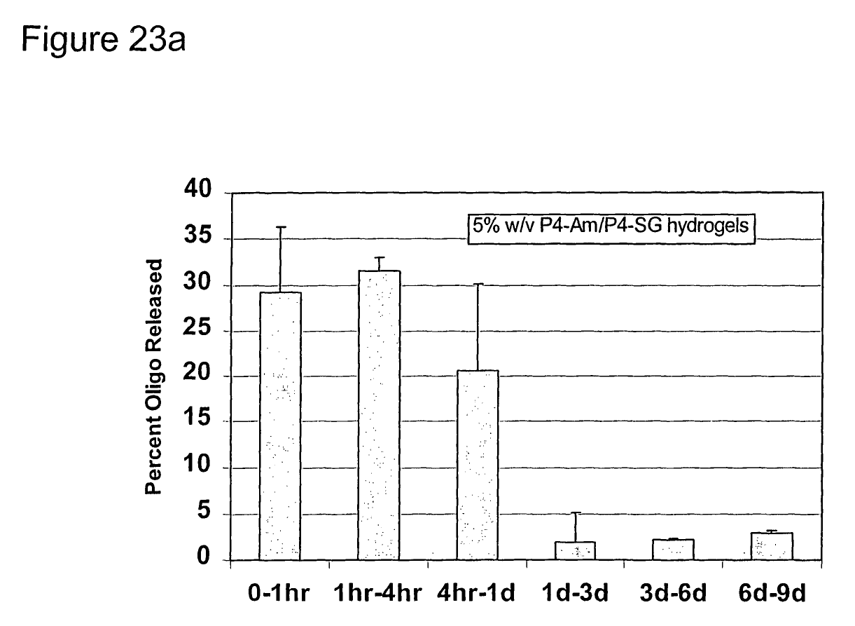

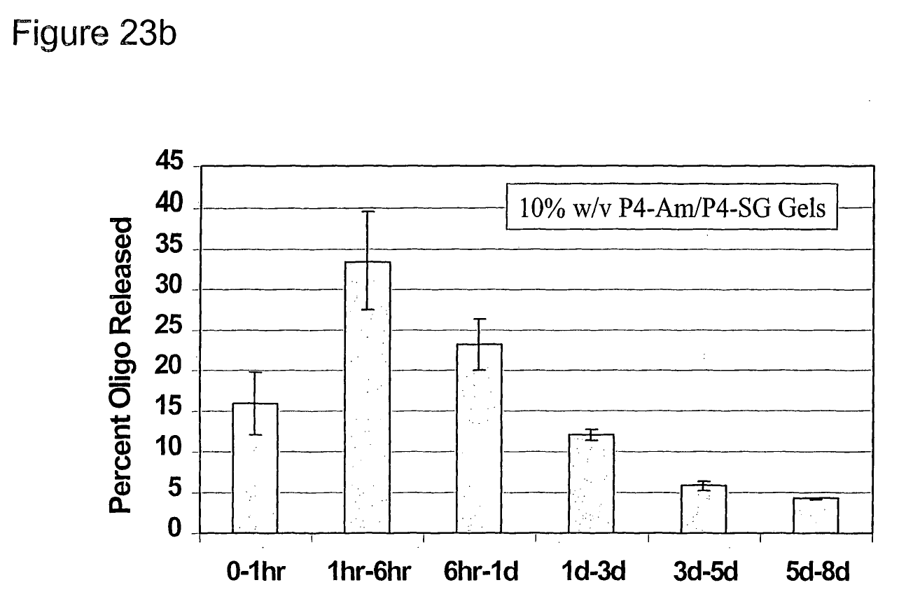

FIGS. 23A and 23B show the results of in-vitro release assays that were performed for 5% and 10% hydrogels containing 1 μg/ml of oligo.

### Example 13

### P4-SH/P4-SG Networks Containing Oligonucleotides

Materials

Polyethylene oxide Tetrasulfydryl (P4-SH) (SunBio Systems)

Poly(ethylene oxide 3350)-tetrasuccinimidyl glutarate (P4-SG) (SunBio Systems)

Oligonucleotides with phosphorothioate or phosphodiester backbones (Oligos, etc.)

Formulations

50 μl of a solution of P4-SG (5% w/v in mQ water) and oligophosphorothioate (100 μg) was mixed with 50 μl of 5% w/v P4-AM (in phosphate buffer, pH 8.0) solution and incubated at 37° C. Additional formulations with 3%, 4%, 10%, 20%, and 30% w/v total PEGs containing oligophosphorothioate were also generated and the formation of a gel was noted in each case.

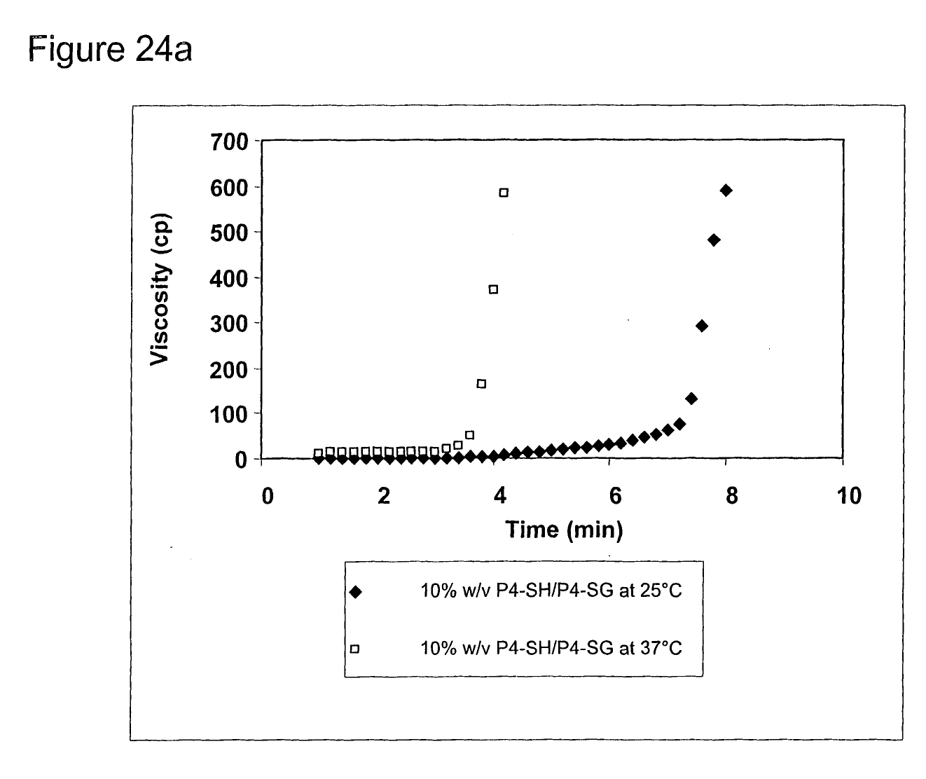

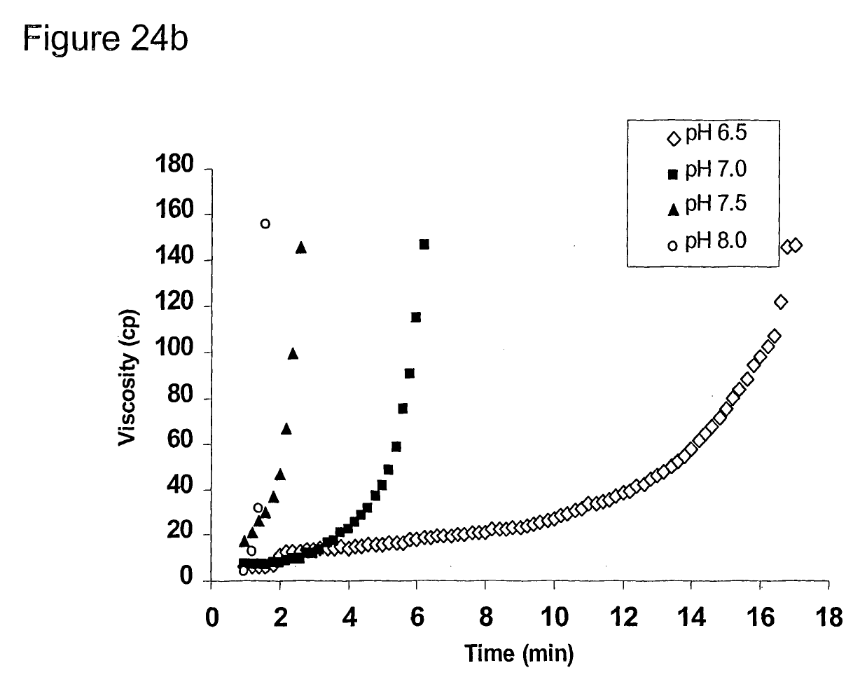

The kinetics of cross-linking of the hydrogels (3%, 4%, 10%, 20%, 30%) was measured by Brookefield Rheometry at 25° C. For each of these formulations, the onset of “gel” formation was characterized by the rapid increase in shear viscosity that marked the critical gel point, G_{c}. Gel times for 20 and 30% w/v gels were less than 2 minutes at 25° C. and 37° C. FIG. 24A demonstrates that at 37° C., the rate of gelation was faster. FIG. 24B demonstrates that the gel time at higher pHs was faster and thus, the gel time could be modulated by variations in temperature and pH.

In Vitro Release of Oligonucleotides

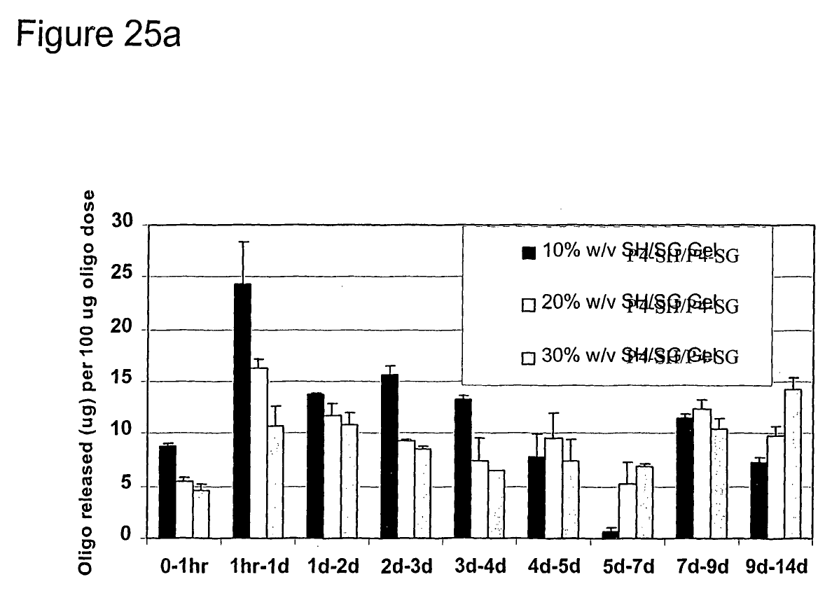

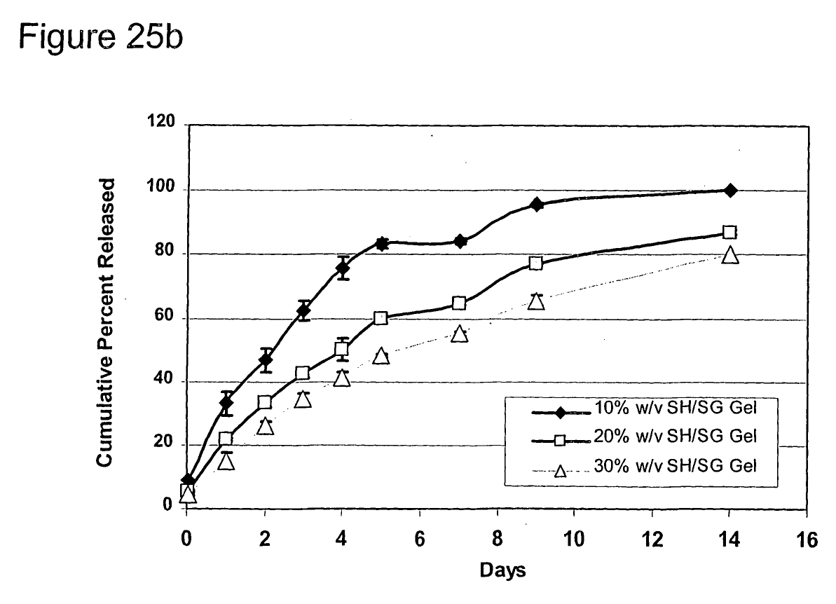

100 μl of a solution of P4-SG in milliQ water was mixed with 100 μl of a solution of P4-SH and 1 μg/ml of oligophosphorothioate (in phosphate buffer, pH 8.0) in a 1.5 ml centrifuge tube and incubated at 37° C. After 1 hour, the gel was retrieved and placed in a new centrifuge tube with 1 ml of phosphate buffered saline, pH 7.4. The gels were incubated at 37° C. At each timepoint, 800 μl of supernatant was retrieved and transferred to a new tube. 800 μl of fresh buffer was added to the tube containing the gel. The supernatant was analyzed for oligophosphorothioate content by anionic exchange chromatography. Release assays were performed for 10, 20 and 30% hydrogels containing 1 μg/ml of oligo. FIGS. 25A and 25B show that in 14 days, the total % ODN released was ˜98% for 10% gels, ˜85% for 20% gels, and ˜78% for 30% gels.

### Example 14

### PAMAM/P4-SG Networks Containing Oligonucleotides

Materials

Poly(amidoamine), Generation 0 (G0), 4 amine groups (Dendritech)

Poly(ethylene oxide 3350)-tetrasuccinimidyl glutarate (P4-SG) (SunBio Systems)

Oligonucleotides with phosphorothioate or phosphodiester backbones (Oligos, etc.)

Network Formulations

50 μl of a solution of P4-SG (10% w/v in mQ water) and oligophosphorothioate (100 μg) was mixed with 50 μl of 0.1% w/v PAMAM, Generation 0 (in phosphate buffer, pH 8.0) solution and incubated at 37° C. The gel time was 4 minutes at 25° C.

In Vitro Release of Oligonucleotides

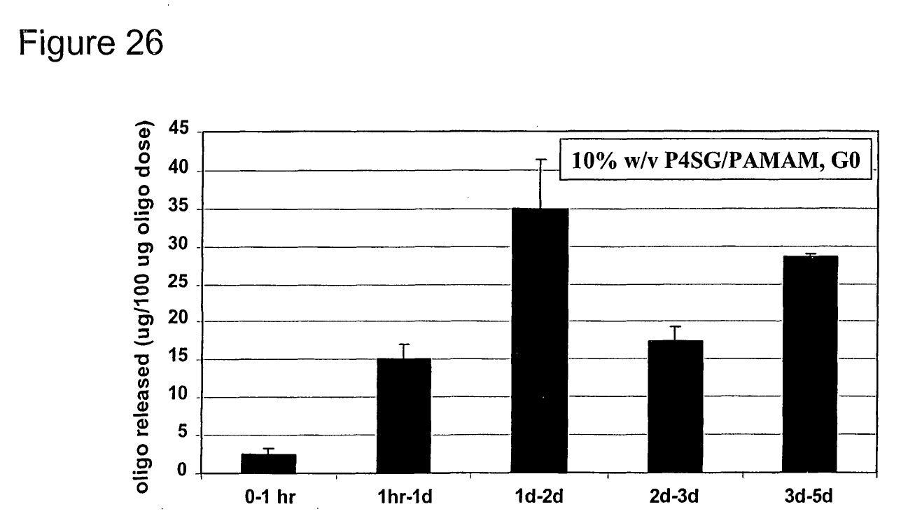

100μl, of a solution of 10% w/v P4-SG in milliQ water was mixed with 100 μl of a solution of 0.5% w/v PAMAM and 1 μg/μl of oligophosphorothioate (in phosphate buffer, pH 8.0) in a 1.5 ml centrifuge tube and incubated at 37° C. After 1 hour, the gel was retrieved and placed in a new centrifuge tube with 1 ml of phosphate buffered saline, pH 7.4. The gels were incubated at 37° C. At each time point, 800 μl of supernatant was retrieved and transferred to a new tube. To the tube containing the gel, was added 800 μl of fresh buffer. The supernatant was analyzed for oligophosphorothioate content by anionic exchange chromatography. FIG. 26 demonstrates that approximately ˜18% of the oligophosphorothioate was released within 1 day and 98.5% was released within 5 days.

### Example 15

### Micronized Calcium Phosphate Oligonucleotide in P4-AM/P4-SG Networks

Materials

CaCl₂: 0.1 M solution in deionized water (Sodium- and potassium-free calcium chloride must be used) (Sigma)

Polyethylene oxide Tetrasulfydryl (P4-SH) (SunBio Systems)

Poly(ethylene oxide 3350)-tetrasuccinimidyl glutarate (P4-SG) (SunBio Systems)

Oligonucleotides with phosphorothioate or phosphodiester backbones (Oligos, etc.)

Formulations

10 μl of 0.1 M CaCl₂ was added dropwise to a 100 μl solution of oligophosphorothioate in deionized water (1 mg/ml), while stirring. A fine white precipitate formed in the tube.

The white precipitate was dialyzed by centrifugation/filtration using a 1.5 ml Centricon Filtrion® centrifuge tube.

The white precipitate was reconstituted in a 3% w/v solution of P4-AM.

50 μl of the P4-AM/OligoCaP dispersion was added to 50 μl of a 3% w/v P4-SG solution to form a 3% total PEGs formulation. Gel time of a 3% PEGs gel with micronized CaP-ODN was ˜10 minutes at 37° C., and 19 minutes at 25° C.

The gel was characterized as a “hard” gel.

### Example 16

### Networks Containing Microparticles in Hydrogel

Materials

Poly(lactide-co-glycolide) microparticles (12,000 Daltons) containing plasmid DNA (Aldveron, LLC)

Poly(ethylene oxide 3350)-tetrasuccinimidyl glutarate (P4-SG) (SunBio Systems)

Poly(ethylene oxide) tetrasulfydryl (P4-SH) (SunBio Systems)

Formulation

10, 50, and 100 mg batches of DNA-containing microparticles were added to 50 μl solutions of 10% w/v P4-SH (A, B, C, respectively) made up in phosphate buffer, pH 8.0. 50 μl of a solution of 10% w/v P4-SG made up in DI water was added to solutions A, B and C to make formulations A, B and C. Gel times and gel characteristics were determined.

Network Characterization

Formulations A, B and C all gelled after between 2-3 minutes at room temperature, demonstrating no inhibition of gelation by addition of microparticles. Hydrogels fabricated from formulation C were found to be hard and brittle. Hydrogels from A and B were hard, but pliable. This study demonstrates the feasibility of incorporation of microparticles into hydrogels for the purpose of applying drug delivery devices to rounded tissues and surfaces. The hydrogel in this case would hold the microparticles “in place.”

### Example 17

### Chitosan/P4-SG Networks (CH/P4-SG)

Materials

Chitosan, glutamate salt (Pronova)(CH), MW ˜1 million

Poly(ethylene oxide 3350)-tetrasuccinimidyl glutarate (P4-SG) (SunBio Systems)

Plasmid DNA, SEAP (Gene Therapy Systems)

Formulation

A solution containing 0.05% chitosan glutamate (CH) was prepared in phosphate buffer, pH 8.0. 50 μl of this solution was added to 50 μl of a solution containing 5% w/v P4-SG and 1 μg/μl DNA in DI water (CH/P4-SG).

Network Characterization: Gel Time, Hardness/Softness

The formulation gelled instantaneously at 25° C., forming a hard gel. This formulation demonstrates the feasibility of a proteolytically degradable network (e.g., a network degradable by lysozymes).

### Example 18

### Poly(lysine)/P4-SG Networks (PL/P4-SG)

Materials

Poly(lysine) hydrobromide, MW ˜150,000 (Sigma)

Poly(ethylene oxide 3350)-tetrasuccinimidyl glutarate (P4-SG) (SunBio Systems)

Plasmid DNA, SEAP (Gene Therapy Systems)

Formulation

A solution containing 1.0% w/v poly(lysine) hydrobromide (PL) was prepared in phosphate buffer, pH 8.0. 50 μl of this solution was added to 50 μl of a solution containing 5% w/v P4-SG and 1 μg/μl DNA in DI water.

Network Characterization: Gel Time, Hardness/Softness

The formulation gelled in between 2 and 3 minutes, and formed a semi-hard gel. This formulation is another variation of a network formulation that can be used for nucleic acid delivery.

### Example 19

### (PEO-PPO-PEO-tetra-SH)/P4-SG Networks (PEO-PPO-PEO/P4-SG)

Materials

Poly(ethylene oxide)-poly(propylene oxide)-poly(ethylene oxide)-tetrasulfhydryl, MW 10K Daltons (PEO-PPO-PEO-tetra-SH) (SunBio Systems)

Poly(ethylene oxide 3350)-tetrasuccinimidyl glutarate (P4-SG) (SunBio Systems)

Formulation 1A solution containing 10% w/v PEO-PPO-PEO-tetra-SH was prepared in phosphate buffer, pH 8.0. 50 μl of this solution was added to 50 μl of a solution of 10% w/v P4-SG and 1 μg/μl DNA in DI water to form a 10% w/v gel.

Network Characterization: Gel Time, Hardness/Softness

The formulation gelled in 6-7 minutes, and formed a hard, oily gel. This formulation is yet another variation of a network formulation that can be used for nucleic acid delivery.

Other Embodiments

It is to be understood that while the invention has been described in conjunction with the detailed description thereof, that the foregoing description is intended to illustrate and not limit the scope of the appended claims. Other aspects, advantages, and modifications are within the scope of the following claims.

What is claimed is:

1. An injectable aqueous formulation, comprising:
a nucleic acid;
a first non-nucleic acid, water-soluble component; and
a second non-nucleic acid, water-soluble component,
wherein the first and second components each include two or more reactive groups, the reactive groups of the first component being reactive with the reactive groups of the second component.

2. The formulation of claim 1, wherein the first and second components react with one another to form a branched or a crosslinked polymeric network.

3. The formulation of claim 1, wherein at least one of the first and second components includes one or more reactive groups selected from the group consisting of succinimidyl, chloroformate, acrylate, amino, alcohol, tetrathiol, epoxide, sulfhydryl, and hydrazidyl groups.

4. The formulation of claim 1, wherein at least one of the first and second components is a functionalized multi-armed poly(alkylene oxide).

5. The formulation of claim 1, wherein one of the first and second components is polyethylene glycol tetraamine.

6. The formulation of claim 1, wherein one of the first and second components is polyethylene glycol tetrasuccinimidyl glutarate.

7. The formulation of claim 1, wherein at least one of the first and second components is a functionalized poly(alkylene oxide) with at least two reactive functional groups.

8. The formulation of claim 1, wherein one of the first and second components is a polyamidoamine having 4 to 8 reactive functional groups.

9. The formulation of claim 1, wherein at least one of the first and second components is a polyethylimine or polylysine derivative.

10. The formulation of claim 1, wherein at least one of the first and second components is a functionalized chitosan, cyclodextrin, or poly(vinyl alcohol) with at least two reactive functional groups.

11. The formulation of claim 1, wherein one or both of the first and second components includes three or more reactive groups, the reactive groups of the first component being reactive with the reactive groups of the second component.

12. The formulation of claim 1, further comprising a third non-nucleic acid, water-soluble component, wherein the third component includes at least one reactive group, the reactive group being reactive with at least one reactive group of the first component, with at least one reactive group of the second component, with at least one reactive group of each of the first and second components, or with at least one reactive group of the product formed by reacting the first and second components.

13. The formulation of claim 1, further comprising methoxy-polyethylene glycol-di-stearoyl-phosphatidylethanolamine (PEG-DSPE).

14. The formulation of claim 1, further comprising an excipient.

15. The formulation of claim 1, wherein the formulation comprises more than one species of nucleic acid.

16. The formulation of claim 1, wherein the nucleic acid is an oligonucleotide.

17. The formulation of claim 1, wherein the nucleic acid encodes a therapeutic protein or a protein that induces an immune response.

18. The formulation of claim 1, wherein the nucleic acid is in a solution, dispersion, or emulsion.

19. The formulation of claim 1, wherein the nucleic acid is encapsulated in a biodegradable polymeric microsphere.

20. The formulation of claim 2, wherein the nucleic acid is released from the branched or crosslinked polymeric network by biodegradation or by simple diffusion.

21. The formulation of claim 1, wherein said formulation forms a hydrogel at a temperature between about 20° C. and about 40° C. within about 20 minutes after said formulation is prepared.

22. The formulation of claim 1, wherein the formulation remains injectable for at least fifteen seconds after said formulation is prepared.

23. The formulation of claim 21, wherein the formulation remains injectable for at least fifteen seconds after said formulation is prepared.

24. The formulation of claim 2, wherein the network forms a viscous liquid.

25. The formulation of claim 2, wherein release of the nucleic acid following injection is controlled by the cross-linking density of the network.

26. The formulation of claim 2, wherein expression of the nucleic acid following injection is controlled by the cross-linking density of the network.

27. The formulation of claim 1, wherein the first and second components are biodegradable.

28. The formulation of claim 2, wherein the network is biodegradable.

29. The polymeric network of claim 2, wherein the branched or crosslinked polymeric network comprises linkages selected from the group consisting of ester, carbonate, imino, hydrazone, acetal, orthoester, peptide, amide, urethane, urea, amino, oligonucleotide, and sulfonamidyl bonds.

30. The formulation of claim 27, wherein the first and second components are biodegradable by a hydrolytic or proteolytic mechanism.

31. The formulation of claim 2, wherein the network is partially crosslinked.

32. The formulation of claim 2, wherein the network is fully crosslinked.

33. The formulation of claim 27, wherein the components comprise one or more functional groups selected from the group consisting of sulfhydryl, amine, epoxide, phosphoroamidite, chloroformate, acrylate, carboxylic acid, aldehyde, succinimide ester, succinimide carbonate, maleimide, iodoacetyl, carbohydrate, isocyanate, and isothiocyanate groups.

34. The formulation of claim 1, wherein at least one of the first and second components comprises a biodegradable linkage selected from the group consisting of lactates, caproates, methylene carbonates, glycolates, ester-amides, ester-carbonates, and combinations thereof.

35. The formulation of claim 14, wherein the excipient is selected from the group consisting of neutral, anionic, and cationic lipids.

36. The formulation of claim 14, wherein the excipient is selected from the group consisting of polyethylene glycol, chitosan, hyaluronic acid, chrondoitin sulfate, heparan sulfate, phosphatidyl inositol, glucosamine, polyvinyl alcohol, pluronics, derivatized pluronics, and derivatized polyethylene glycol.

37. The formulation of claim 14, wherein the excipient comprises a permeation enhancer.

38. The formulation of claim 14, wherein the excipient comprises a bioavailability enhancer.

39. The formulation of claim 14, wherein the excipient is a cytokine.

40. The formulation of claim 14, wherein the excipient is a small molecule drug.

41. The formulation of claim 14, wherein the excipient is chemically bound to the crosslinked polymeric network or branched polymer.

42. A method of making a polypeptide, the method comprising applying the formulation of claim 1 to a cell, wherein the nucleic acid codes for expression of the polypeptide.

43. The method of claim 42, wherein the formulation is applied to a cell within an animal.

44. The method of claim 43, wherein the formulation is administered to the animal by injection, extrusion, or spraying.

45. A method of making a polypeptide, the method comprising injecting into an animal the formulation of claim 1, wherein the nucleic acid codes for expression of the polypeptide.

46. The method of claim 45, wherein the formulation is injected in, on, or adjacent to a tumor.

47. The method of claim 45, wherein the formulation is injected intra-joint.

48. The method of claim 45, wherein the formulation is injected into the animal more than once.

49. The method of claim 45 wherein the formulation of claim 1 is premixed before injection.

50. The method of claim 45, wherein the animal is a human.

51. A method of producing a polypeptide, the method comprising:
providing a surface suitable for cell culture;
adding the formulation of claim 1 to the surface; and
placing a cell on the formulation,
wherein the nucleic acid codes for expression of the polypeptide, and wherein the cell produces the polypeptide following the culturing of the cell in vitro.

52. A method of making a nucleic acid-containing microparticle preparation, the method comprising:
introducing the nucleic acid and the first and second non-nucleic acid components of the formulation of claim 1 into an emulsifying bath; and
emulsifying the resulting mixture during at least part of the time that said first and second non-nucleic acid, water-soluble components are reacting with each other, to result in microparticles containing said nucleic acid molecules.

53. The method of claim 52, wherein said stirring is sufficiently vigorous so as to result in microparticles having an average diameter of less than about 500 microns.

54. The method of claim 52, wherein said emulsifying is sufficiently vigorous so as to result in microparticles having an average diameter of less than about 250 microns.

55. The method of claim 52, wherein said emulsifying is sufficiently vigorous so as to result in microparticles having an average diameter of less than about 100 microns.

56. The method of claim 52, wherein said emulsifying is sufficiently vigorous so as to result in microparticles having an average diameter of less than about 50 microns.

57. The method of claim 52, wherein said emulsifying is sufficiently vigorous so as to result in microparticles having an average diameter of less than about 20 microns.

58. The method of claim 52, wherein said emulsifying is sufficiently vigorous so as to result in microparticles having an average diameter of less than about 15 microns.

59. The method of claim 52, wherein said emulsifying is sufficiently vigorous so as to result in microparticles having an average diameter of less than about 10 microns.

60. The method of claim 52, wherein said emulsifying is sufficiently vigorous so as to result in microparticles having an average diameter of less than about 5 microns.

61. The method of claim 52, wherein said emulsifying is sufficiently vigorous so as to result in microparticles having an average diameter of less than about 1 microns.

62. The method of claim 52, wherein said introducing step comprises coextruding said first and second components into an aqueous solution in the emulsifying bath.

63. The formulation of claim 1, wherein said formulation comprises microparticles.

64. A method of making a dried nucleic acid formulation comprising:
(a) preparing a mixture by mixing in an aqueous solution
(i) a nucleic acid,
(ii) a first non-nucleic acid, water-soluble component,
(iii) a second non-nucleic acid, water-soluble component, and
(iv) a third non-nucleic acid, water-soluble component,
wherein the first and second components each include two or more reactive groups, the reactive groups of the first component being reactive with the reactive groups of the second component at a pH greater than 7.0, and
wherein the aqueous solution has a pH and temperature that prevents the first and second components from reacting to form a cross-linked network; and
(b) drying the mixture to thereby create a dried nucleic acid formulation.

65. The method of claim 64, wherein the mixing is performed at a pH less than about 7.0.

66. The method of claim 65, wherein the mixing is performed at a pH less than about 6.0.

67. The method of claim 66, wherein the mixing is performed at a pH of about 5.5.

68. The method of claim 64, wherein the mixing is performed at or below about 4° C.

69. The method of claim 64, wherein the mixture is lyophilized.

70. The method of claim 64, wherein the first non-nucleic acid, water-soluble component is polyethylene glycol amine.

71. The method of claim 64, wherein the second non-nucleic acid, water-soluble component is polyethylene glycol succinimidyl glutarate.

72. The method of claim 64, wherein the third non-nucleic acid, water-soluble component is methoxy-polyethylene glycol-di-stearoyl-phosphatidylethanolamine (PEG-DSPE).

73. A method of preparing a nucleic acid-containing formulation, the method comprising adding a buffer having a pH greater than 7.0 to the dried nucleic acid formulation of claim 64, wherein the addition of the buffer results in the formation of a crosslinked network between the first and second components.

74. The method of claim 73, wherein the buffer is a phosphate buffer and has a pH of about 7.5.

75. The method of claim 73, wherein the adding step is performed at or above 20° C.

76. The method of claim 75, wherein the adding step is performed at or above 37° C.

77. The method of claim 64, wherein the third component includes at least one reactive group that is reactive at a pH greater than 7.0 with at least one reactive group of the first component, with at least one reactive group of the second component, with at least one reactive group of each of the first and second components, or with at least one reactive group of the product formed by reacting the first and second components.

78. A dried formulation comprising:
(a) a nucleic acid;
(b) a first non-nucleic acid, water-soluble component;
(c) a second non-nucleic acid, water-soluble component; and
(d) a third non-nucleic acid, water-soluble component,
wherein the first and second components each include two or more reactive groups, the reactive groups of the first component being reactive with the reactive groups of the second component,
wherein the first and second components are in an unreacted state, and wherein the nucleic acid and the three components are not in solution.

79. The formulation of claim 78, wherein the formulation is lyophilized.

80. The formulation of claim 78, wherein the first non-nucleic acid, water-soluble component is polyethylene glycol amine.

81. The formulation of claim 78, wherein the second non-nucleic acid, water-soluble component is polyethylene glycol succinimidyl glutarate.

82. The formulation of claim 78, wherein the third non-nucleic acid, water-soluble component is methoxy-polyethylene glycol-di-stearoyl-phosphatidylethanolamine (PEG-DSPE).

83. A kit comprising:
the formulation of claim 78; and
a buffer having a pH of at least 7.0.

84. A method of administering a nucleic acid to an individual the method comprising:
preparing a mixture by adding a buffer having a pH of at least 7.0 to the formulation of claim 78;
incubating the mixture to permit the formation of a crosslinked network; and
administering the mixture to the individual.

85. The formulation of claim 78, wherein the third component includes at least one reactive group that is reactive at a pH greater than 7.0 with at least one reactive group of the first component, with at least one reactive group of the second component, with at least one reactive group of each of the first and second components, or with at least one reactive group of the product formed by reacting the first and second components.

86. A method of delivering a particle to an individual, the method comprising:
administering to the individual a formulation comprising said particle; a first, non-nucleic acid, water soluble component; and a second, non-nucleic acid, water soluble component, wherein the first and second components each include two or more reactive groups, the reactive groups of the first component being reactive with the reactive groups of the second component.

87. The method of claim 86, wherein the particle is a virus or viral particle.

88. The method of claim 86, wherein the particle is an adenovirus or adenoviral particle.
